(19) United States
(12) Patent Application Publication
(10) Pub. No.: US 2004/0115207 A1
(43) Pub. Date: Jun. 17, 2004

(54) Bioconjugates and uses thereof

(76) Inventors: Bernstein D Irwin, Seattle, WA (US); Peter D Senter, Seattle, WA (US); Craig Cano Beeson, Charleston, SC (US); Michael Hart, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/181,311

(22) PCT Filed: Jan. 11, 2001
(86) PCT No.: PCT/US01/01153

(30) Foreign Application Priority Data
Dec. 9, 1999 (DK) BA 1999 00439

(51) Int. Cl. A61K 51/00; A61K 39/395; C07K 16/46

(52) U.S. Cl. 424/178.1; 424/1.49; 530/391.1

(57) ABSTRACT

A novel bioconjugate and a method for delivering the bioconjugate to a cell site is described. In particular, the bioconjugate composition comprises a targeting agent conjugated to a diagnostically or therapeutically effective agent by a metabolizable linker moiety which is cleaved by an exogenous enzyme.

### TECHNICAL FIELD

The invention relates generally to novel bioconjugates and a method for delivering these bioconjugates to a cell site. In particular, the present invention relates to a bioconjugate composition comprising a targeting agent conjugated to a diagnostically or therapeutically effective agent by a metabolizable linker moiety which is cleaved by an exogenously administered enzyme.

### BACKGROUND OF THE INVENTION

Targeting agents such as antibodies and antibody fragments have been used for the selective/targeted delivery of therapeutic agents to a target-specific site. For example, in cancer chemotherapy, an anti-cancer drug may be conjugated to a targeting agent such as a tumor-specific antibody that is complementary to a tumor-specific antigen. The drug is released from the conjugate at the tumor cells, where it exerts its toxic effects on the target cells. Therapeutic agents generally used in these targeting systems include radioisotopes; drugs such as adriamycin, vincristine, cisplatin, doxorubicin, daunomycin, methotrexate, cyclophosphanmide and isophosphamide and mitomycin C; toxins such as diphtheria toxin, pseudomonas toxin and ricin; and anti-tumor drugs such as used in cancer chemotherapy.

However, these delivery systems have several disadvantages. Traditional methods for direct attachment of therapeutic agents to antibodies involves linkers that are highly stable under physiological conditions. This stability, while a necessary feature, results in biodistribution and whole body clearance of the therapeutic agent that is dependent on the properties of the monoclonal antibody. As such, only a small fraction of the therapeutic agent is delivered to the tumor mass and the majority of the conjugate remains in circulation for extended periods of time. This can lead to dose-limiting toxicities.

Alternate approaches have been devised to improve the radioisotope biodistribution by using a pre-targeting mechanism. These strategies generally involve the administration of a non-radiolabeled monoclonal antibody conjugate. Time is permitted for the unbound antibody to be cleared before a radioisotope complex designed to bind to the antibody conjugate that is bound to the target epitope. This approach permits rapid systemic clearance of the radioisotope. In one strategy three steps are required to achieve tumor targeting. In this method, an antibody streptavidin conjugate is administered and is allowed to localize within a solid tumor mass. The conjugate is cleared from the system using a biotinylated clearing agent that is taken up in the liver and kidneys. The final step involves the administration of a biotinylated radionuclide that binds to the antibody-streptavidin complex on the tumor mass. Significant drawbacks to this strategy include the complexity of the approach, the fact that the streptavidin-monoclonal antibody complex can be blocked by endogenous biotin present in the patient as well as the biotinylated clearing agent and that streptavidin is immunogenic requiring therapeutic efficacy in the first course of therapy.

In another delivery system, a therapeutic agent is conjugated to a biodegradable polyamino acid macromolecular carrier that may in turn be linked to a targeting agent. Degradation of the polyamino acid carrier in the target cells releases the cytotoxic drug. However, polyamino acid carriers suffer from problems similar to those associated with the use of antibodies as drug carriers. For example, bulky polyamino acid carriers may reduce the ability of the conjugate to internalize within the cell. Antibody-enzyme conjugates have been used to amplify antibody-mediated cytotoxicity. (See, e.g., U.S. Pat. No. 4,975,278 and Canadian Patent No. 1,216,791).

Targeting agents conjugated to a moiety containing a substrate for an enzyme have also been used as a delivery system. For example, monoclonal antibodies (mAb) can be used as targeting agents for an enzyme that can generate cytotoxic drugs from non-cytotoxic precursors (prodrugs) within tumor masses. Generally, the enzyme is conjugated to the targeting agent, and the prodrug is administered either simultaneously or subsequently. However, these prodrugs may be activated by plasma or other normal tissues prior to reaching the target site. Additionally, the targeted enzymes are generally of microbial origin and can themselves be potentially immunogenic in humans. Radiolabeled antibody therapy, wherein the radiolabeled antigen is conjugated to a moiety containing a substrate for an endogenous enzyme, can be used to reduce nonspecific radiation delivery. (See Studer M. et al., Bioconjugate Chem., 3:424-429, 1992; Stein R. et al., Journal of Nucl Med, 38:1392-1400, 1997; DeNardo G. L. et al., Clin Can Res, 4:2483-2490, 1998; Arano Y. et al., Bioconjugate Chem, 2:497-506, 1998). For example, conjugates containing substrates preferentially catabolized in the liver by cathepsin G have been used to conjugate antibody with metal chelates, to decrease the amount of the radioisotope in liver. (Studer M. et al., Bioconjugate Chem., 3:424-429, 1992 and DeNardo G. L. et al., Clin Can Res, 4:2483-2490, 1998). However, a major drawback to this approach is that the reduction of background radiation is limited to a particular organ.

Therefore, current delivery systems have several disadvantages. Thus, there is a need for improved compositions and methods for delivering therapeutic diagnostic agents to a predetermined site while increasing retention of the agent at the site and increasing clearance of the agent from the circulation.

### SUMMARY OF THE INVENTION

The present invention addresses the aforementioned needs in the art by providing a bioconjugate composition comprising a targeting agent conjugated to a diagnostically or therapeutically effective agent by a metabolizable linker moiety which is cleaved by an exogenous enzyme. The enzyme cleaves the metabolizable linker moiety to release the therapeutic/diagnostic agent at the target site.

In one aspect, the invention relates to a bioconjugate composition comprising a targeting agent conjugated to a diagnostically or therapeutically effective agent by a metabolizable linker moiety, such as, but not limited to a β-lactamase-sensitive linker moiety. The targeting agent may be an antibody and the diagnostically or therapeutically effective agent can be a radioisotope. Preferably the targeting agent is an antibody or a fragment thereof. Within one preferred embodiment the targeting agent is a monoclonal antibody. In a preferred embodiment, the antibody is an anti-CD19 antibody, an anti-CD20 antibody, an anti-CD22 antibody, an anti-CD33 antibody, an anti-CD37 antibody, an anti-CD45 antibody or any cell surface receptor, and the diagnostically or therapeutically effective agent is Cu-64, Ga-67, Ga-68, Zr-89, Ru-97, Tc-99m, Rh-105, Pd-109, In-111, I-123, I-125, I-131, Y-90, Re-186, Re-188, Au-198, Au-199, Pb-203, At-211, Pb-212 and Bi-212.

In another aspect, the invention relates to a bioconjugate composition comprising the formula (I):

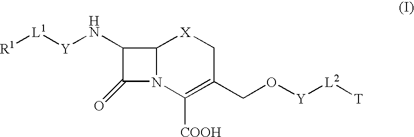

wherein m is an integer ranging from 1 to 12 inclusive; and n is an integer ranging from 1 to 12 inclusive;

L¹ is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —(CHR²)ₙ—CH₂—O—; —(CHR²)ₙ—; —NH—(CHR²)ₙ—NH—; —NH—(CHR²)ₙ—NH—CO—(CHR²)ₘ—CO-Z-; —(CHR²)ₙ—NH—CS—NH—(CHR²)ₘ—CS—NH-Z; —NH—(CHR²)ₙ—NH—CS—(CHR²)ₘ—CO-Z-; —(CHR²)ₙ—NH—CO—NH—(CHR²)ₘ—CO—NH-Z; or a biodegradable polyamino acid macromolecular carrier, wherein L¹-Y—NH taken together optionally form a heterocyclic or a heteroaryl ring;

L² is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —NH—(CHR²)ₙ—NH—; —NH—(CHR²)ₙ—(CHR³)—NH—; —NH—(CHR²)ₙ—NH—CO—(CHR²)ₘ—CO-Z-; —NH—(CHR²)ₙ—NH—CO—(CHR²)ₘ—CO—; —NH—(CHR²)ₙ—NH—CS—(CHR²)ₘ—CO-Z-; —NH—(CHR²)ₙ—NH—CS—(CHR²)ₘ—CO—; —(CHR²)ₙ—CH₂—O—; —(CHR²)ₙ—; or a biodegradable polyamino acid macromolecular carrier; wherein L² optionally forms cyclic structure comprising an aryl ring, heteroaryl ring, cycloalkyl ring, cycloalkenyl ring, wherein said ring is optionally substituted;

T is a targeting agent;

X is O, NH, S or SO;

Y is CO or CS;

Z is an amino acid, N-hydroxysuccinimydl (NHS) or sulfonated N-hydroxysuccinimydl;

R¹ is a diagnostically or therapeutically effective agent;

R² is H, OH, lower alkyl, alkoxy, acyloxy, alkylamino, alkylthio or hydroxyalkyl;

R³ is —COOH or —CH₂OSO₃H; or

a pharmaceutically acceptable salt thereof.

In another aspect, the invention relates to a bioconjugate composition comprising the formula (II):

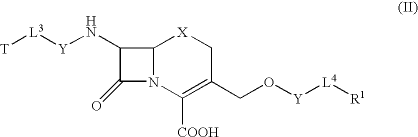

wherein m is an integer ranging from 1 to 12 inclusive; and n is an integer ranging from 1 to 12 inclusive;

L³ is —(CHR²)ₙ—NH—(CHR²)ₘ—CO-Z; —(CHR²)ₙ—NH—CO—(CHR²)ₘ—CO-Z; —(CHR²)ₙ—CO—NH-Z; —(CHR₂)ₙ—NH—; —(CHR²)ₙ—NH—CO—NH—(CHR²)ₘ—CO—NH-Z-; —(CHR²)ₙ—CH₂—S—; —(CHR²)ₙ—CH₂—O—; —NH—(CHR²)ₙ—NH—CS—(CHR²)ₘ—CO-Z; —NH—(CHR²)ₙ—NH—; —NH—(CHR²)ₙ—NH—CO—(CHR²)ₘ—CO-Z; —(CHR₂)ₙ—; —(CHR²)ₙ—NH—CS—NH—(CHR²)ₘ—CS—NH-Z; or a biodegradable polyamino acid macromolecular carrier, wherein L³-Y—NH taken together optionally form a heterocyclic or a heteroaryl ring;

L⁴ is —(CHR²)ₙ—NH—(CHR²)ₘ—CO-Z; CHR²)ₙ—NH—CO—(CHR²)ₘ—CO-Z; —(CHR²)ₙ—NH—; —(CHR²)ₙ—CH₂—S—; —NH—(CHR²)ₙ—NH—CO—(CHR²)ₘ—CO-Z-; —(CHR²)ₙ—CH₂—O—; —(CHR²)ₙ—; —NH—(CHR²)ₙ—NH—; —NH—(CHR²)ₙ—(CHR³)—NH—; —NH—(CHR²)ₙ—NH—CO—(CHR²)ₘ—CO—; —NH—(CHR²)ₙ—NH—CS—(CHR²)ₘ—CO-Z-; —NH—(CHR²)ₙ—NH—CS—(CHR²)ₘ—CO—; or a biodegradable polyamino acid macromolecular carrier, wherein L⁴ optionally forms cyclic structure comprising an aryl ring, heteroaryl ring, cycloalkyl ring, cycloalkenyl ring, wherein said ring is optionally substituted;

T is a targeting agent;

X is O, NH, S or SO;

Y is CO or CS;

Z is an amino acid, N-hydroxysuccinimydl (NHS) or sulfonated N-hydroxysuccinimydl;

R¹ is a diagnostically or therapeutically effective agent;

R² is H, OH, lower alkyl alkoxy, acyloxy, alkylamino, alkylthio or hydroxyalkyl;

R³ is —COOH or —CH₂OSO₃H; or

a pharmaceutically acceptable salt thereof.

In a preferred embodiment, the amino acid is selected from the group consisting of lysine, serine, threonine, tyrosine and cysteine; T is an antibody, more preferably an anti-CD19 antibody, an anti-CD20 antibody, an anti-CD22 antibody, an anti-CD33 antibody, an anti-CD37 antibody or an anti-CD45 antibody; and R¹ is a radioisotope, more preferably I-131, iodinated(I-131) aryl glycoside, 5-iodo(I-131)-3-pyridine-carboxylate, Y-90 within metal chelates.

In another preferred embodiment, the invention relates to a bioconjugate composition comprising the formula (I-A)

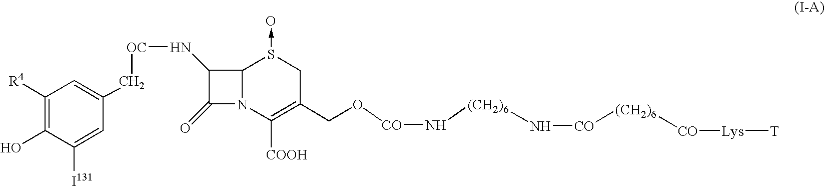

wherein T is an antibody, biotin, streptavidin or avidin; and R⁴ is H or I¹³¹.

In another preferred embodiment, the invention relates to a bioconjugate composition comprising the formula (II-A)

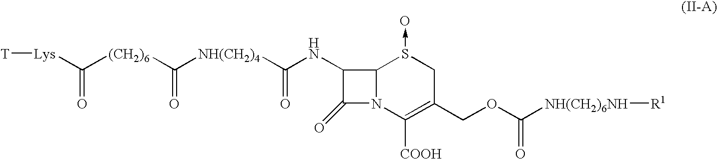

wherein T is an antibody, biotin, streptavidin or avidin; and

R¹ is an iodinated(I-131) aryl glycoside, 5-iodo(I-131)-3-pyridinecarboxyl or Y-90 1,4,7,10-tetraazacyclododecane-N,N′,N″,N′″-tetraacetic acid (DOTA) complex.

In another preferred embodiment, the invention relates to a bioconjugate composition comprising the formula (II-B)

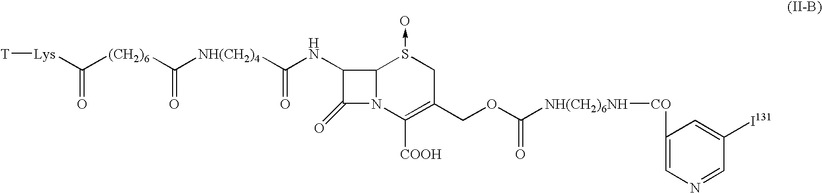

wherein T is an antibody, biotin, streptavidin or avidin.

In another preferred embodiment, the invention relates to a bioconjugate composition comprising the formula (II-C)

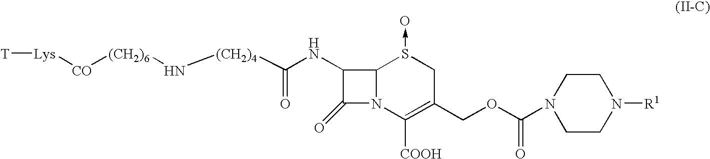

wherein T is an antibody, biotin, streptavidin or avidin; and

R¹ is an iodinated(I-131) aryl glycoside, 5-iodo(I-131)-3-pyridinecarboxyl or Y-90 1,4,7,10-tetraazacyclododecane-N,N′,N″,N′″-tetraacetic acid (DOTA) complex.

In another preferred embodiment, the invention relates to a bioconjugate composition comprising the formula (II-D)

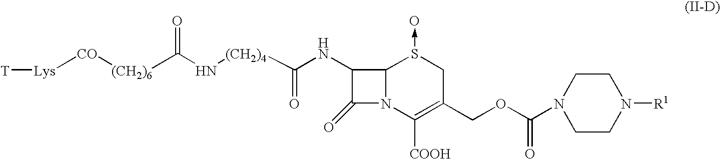

wherein T is an antibody, biotin, streptavidin or avidin; and R¹ is

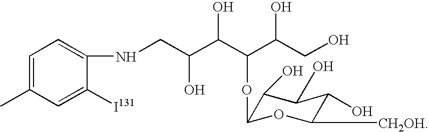

In an alternative embodiment, the invention relates to a method for treating cancer comprising administering to a mammal in need of such treatment a pharmaceutically effective amount of a bioconjugate as described above, and a pharmaceutically effective amount of an enzyme capable of cleaving said metabolizable linkage. In a preferred embodiment, the enzyme is administered subsequent to administration of the bioconjugate.

In an alternative embodiment, the invention relates to a method for the delivery of a diagnostic or a therapeutically effective agent to cells comprising administering a pharmaceutically effective amount of a bioconjugate as described above, wherein the targeting agent is reactive with a binding site on the surface of said cells; and administering a pharmaceutically effective amount of an enzyme capable of cleaving said metabolizable linkage. In a preferred embodiment, the cells are cancer cells. In other preferred embodiments, the enzyme is administered subsequent to administration of the bioconjugate.

In an alternative embodiment, the invention relates to a method of detecting the presence of a disease in a mammal suspected of having said disease, comprising administering to the mammal a diagnostically effective amount of a bioconjugate as described above, and an effective amount of an enzyme capable of cleaving said metabolizable linkage.

These and other embodiments of the present invention will readily occur to those of ordinary skill in the art in view of the disclosure herein.

### BRIEF DESCRIPTION OF THE DRAWINGS

FIG. 1 illustrates flow cytometry depicting binding of intact 1F5 anti-CD20 antibody, 1F5 scFv GS1, and control antibody to Ramos lymphoma cells. The horizontal axis depicts fluorescence intensity of a fluoresceinated goat anti-mouse anti-Ig secondary reagent detecting bound mAb or scFv.

FIGS. 2A and 2B illustrate the time activity curves (±SD) for blood (FIG. 2A) and urine (FIG. 2B), expressed as % ID/g. Solid lines indicate enzyme-treated mice and broken lines indicate control mice not injected with β-lactamase.

FIGS. 3A and 3B illustrate the concentration of radioactivity in tissues expressed as percent of injected dose per gram tissue in mice necroscopsied at 1 h (FIG. 3A) and 20 h (FIG. 3B) post enzyme infusion.

FIG. 4 illustrates the relative concentration of radiolabeled antibody in normal lungs, tumor, and in normal lung following cleavage. These curves represent plots of the effective (i.e. not corrected for radioactive decay) concentration, or percent injected activity per gram of tissue, as a function of time. The area under the effective curve is closely related to total absorbed dose.

### DETAILED DESCRIPTION

The practice of the present invention will employ, unless otherwise indicated, conventional methods of chemistry, biochemistry, pharmacology, molecular biology, microbiology, and recombinant DNA technology, within the skill of the art. Such techniques are explained fully in the literature. See, e.g., Scopes, R. K, Protein Purification Principles and Practices, 2d ed. (Springer-Verlag, 1987); Remington's Pharmaceutical Sciences, 19th Edition (Easton, Pa.: Mack Publishing Company, 1995); Methods In Enzymology (S. Colowick and N. Kaplan, eds., Academic Press, Inc.); Sambrook et al., Molecular Cloning: A Laboratory Manual, 2d ed., Cold Spring Harbor Press, Cold Spring Harbor, N.Y., 1989; Handbook of Experimental Immunology, Vols. I-IV (D. M. Weir and C. C. Blackwell, eds, 1986, Blackwell Scientific Publications); House, Modern Synthetic Reactions, 2nd ed., Benjamin/Cummings, Menlo Park, Calif., 1972.; Fieser and Fieser's Reagents for Organic Synthesis, Wiley & Sons, New York, 1991, Volumes 1-15; Rodd's Chemistry of Carbon Compounds, Elsevier Science Publishers, 1989, Volumes 1-5 and Supplementals; and Organic Reactions, Wiley & Sons, New York, 1991, Volumes 1-40.

All patents, patent applications, and publications mentioned herein, whether supra or infra, are hereby incorporated by reference in their entirety.

It must be noted that, as used in this specification and the appended claims, the singular forms “a”, “an”, and “the” include plural referents unless the content clearly dictates otherwise. Thus, for example, reference to “an antibody” includes two or more such antibodies and the like.

I. Definitions

In describing the present invention, the following terms will be employed, and are intended to be defined as indicated below.

“Lower alkyl” means the monovalent linear or branched saturated hydrocarbon radical, consisting solely of carbon and hydrogen atoms, having from one to six carbon atoms inclusive, unless otherwise indicated. Examples of a lower alkyl radical include, but are not limited to, methyl, ethyl, propyl, isopropyl, n-butyl, isobutyl, tert-butyl,pentyl, n-hexyl and the like.

“Alkoxy” means the radical —O—R, wherein R is a lower alkyl radical as defined above. Examples of an alkoxy radical include, but are not limited to, methoxy, ethoxy, isopropoxy, and the like.

“Acyloxy” means the radical —OC(O)R, wherein R is an alkyl radical as defined above. Examples of acyloxy radicals include, but are not limited to, acetoxy, propionyloxy, and the like.

“Acyl” or “alkanoyl” means the radical —C(O)—R wherein R is an alkyl as defined above. Examples of acyl radicals include, but are not limited to, formyl, acetyl, propionyl, butyryl, and the like.

“Alkylamino” means the radical —NHR or —NR′R″, wherein R′ and R″ are each independently alkyl radicals as defined above. Examples of alkylamino radicals include, but are not limited to, methylamino, (1-ethylethyl)amino, dimethylamino, methylethylamino, diethylamino, di(1-methylethyl)amino, and the like.

“Aminoalkyl” means the radical —RNR′R″, wherein R is an alkyl radical as defined above, and R′ and R″ are each independently H or an alkyl radical as defined above. Examples of aminoalkyl radicals include, but are not limited to, aminomethyl, aminoethyl, aminopropyl, and the like.

“Alkylthio” means the radical —SR, wherein R is an alkyl radical as defined above. Examples of alkylthio radicals include, but are not limited to, methylthio, butylthio, and the like.

“Aryl” means the monovalent monocyclic aromatic hydrocarbon radical consisting of one or more fused rings in which at least one ring is aromatic in nature, which can optionally be substituted with one or more of the following substituents: hydroxy, cyano, alkyl, alkoxy, thioalkyl, halo, haloalkyl, trifluoromethyl, hydroxyalkyl, alkoxycarbonyl, nitro, amino, alkylamino, dialkylamino, aminocarbonyl, carbonylamino, aminosulfonyl and sulfonylamino, unless otherwise indicated. Examples of aryl radicals include, but are not limited to, phenyl, naphthyl, biphenyl, diphenylmethyl, 9H-fluorenyl, indanyl, anthraquinolyl, and the like.

“Heteroaryl” means the monovalent aromatic carbocyclic radical having one or more rings incorporating one, two, or three heteroatoms within the ring (chosen from nitrogen, oxygen, or sulfur) which can optionally be substituted with one or more of the following substituents: hydroxy, cyano, alkyl, alkoxy, thioalkyl, halo, haloalkyl, trifluoromethyl, hydroxyalkyl, alkoxycarbonyl, nitro, amino, alkylamino, dialkylamino, aminocarbonyl, carbonylamino, aminosulfonyl and sulfonylamino, unless otherwise indicated. Examples of heteroaryl radicals include, but are not limited to, naphtyridinyl, anthranilyl, benzooxazolyl, pyridyl, pyrrolyl, pyrazolyl, pyrazinyl, pyrimidyl, thiophenyl, furanoyl, benzofuranoyl, dihydrobenzofuranoyl, 3,3-dimethyl-2,3-dihydrobenzofuranoyl, quinolinyl, isoquinolinyl, tetrahydroquinolinyl, 1,2,3,4-tetrahydroquinolinyl, 1,2,3,4-tetrahydroisoquinolinyl, tetrahydroquinoxalinyl, benzdioxazolyl, benzoisoquinolinyl dione, benzodioxanyl, indolyl, 2,3-dihydroindolyl, thianaphthenyl, dihydrothianaphthenyl, imidazolyl, benzoimidazolyl benzimidazolyl, azabenzimidazolyl, oxazolyl, isooxazolyl, quinoxalinyl, thiazolyl, benzothiazolyl, thiazolidinyl, pyranyl, tetrahydropyranyl pyranyl, benzo[1,3]dioxolyl, 2,3-dihydrobenzo[1,4]dioxinyl, thienyl, benzo[b]thienyl, 1,2,3,4-tetrahydro[1,5]naphthyridinyl, 2H-3,4-dihydrobenzo[1,4]oxazine, 4,5-dihydro-1H-imidazol-2-yl, and the like.

“Cycloalkyl” means the monovalent saturated carbocyclic radical consisting of one or more rings, which can optionally be substituted with one or more of the following substituents: hydroxy, cyano, alkyl, alkoxy, thioalkyl, halo, haloalkyl, trifluoromethyl, hydroxyalkyl, alkoxycarbonyl, nitro, amino, alkylamino, aminocarbonyl, carbonylamino, aminosulfonyl and sulfonylamino, unless otherwise indicated. Examples of cycloalkyl radicals include, but are not limited to, cyclopropyl, cyclobutyl, 3-ethylcyclobutyl, cyclopentyl, cyclohexyl, cycloheptyl, hydrogenated derivatives of aryl as defined above, and the like.

“Cycloalkenyl” means the monovalent unsaturated carbocyclic radical consisting of one or more rings, which can optionally be substituted with one or more of the following substituents: hydroxy, cyano, alkyl, alkoxy, thioalkyl, halo, haloalkyl, trifuoromethyl, hydroxyalkyl, alkoxycarbonyl, nitro, amino, alkylamino, dialkylamino, aminocarbonyl, carbonylamino, aminosulfonyl and sulfonylamino, unless otherwise indicated. Examples of cycloalkenyl radicals include, but are not limited to, cyclopentenyl, cyclohexenyl, cycloheptenyl, hydrogenated derivatives of aryl as defined above, and the like.

“Heterocyclic” means the monovalent saturated carbocyclic radical, consisting of one or more rings, incorporating one, two or three heteroatoms (chosen from nitrogen, oxygen or sulfur), which can optionally be substituted with one or more of the following substituents: hydroxy, cyano, alkyl alkoxy, thioalkyl, halo, haloalkyl, trifluoromethyl, hydroxyalkyl, alkoxycarbonyl nitro, ammo, alkylamino, dialkylamino, aminocarbonyl, carbonylamino, aminosulfonyl and sulfonylamino, unless otherwise indicated. Examples of heterocyclic radicals include, but are not limited to, metabolically inert sugars, such as lactose, cellobiose; tetrahydrofuranoyl tetrahydropyranyl, piperidinyl, piperazinyl, morpholinyl, thiomorpholinyl, 1,1-dioxo-thiomorpholinyl, imidazolidinyl, pyrrolidinyl, pyrrolidin-2-one, pyrrolidin-2,3-dione, hydrogenated derivatives of heteroaryl as defined above, and the like.

“Halogen” means the radical fluoro, chloro, bromo, and iodo.

“Haloalkyl” means the alkyl radical as defined above substituted in any position with one or more halogen atoms as defined above. Examples of haloalkyl radicals include, but are not limited to, 1,2-difluoropropyl, 1,2-dichloropropyl, trifluoromethyl, 2,2,2-trifluoroethyl, 2,2,2-trichloroethyl, and the like.

“Hydroxyalkyl” means the alkyl radical as defined above, substituted with one or more hydroxy groups. Examples of hydroxyalkyl radicals include, but are not limited to, hydroxymethyl, 2-hydroxyethyl, 2-hydroxypropyl, 3-hydroxypropyl, 2-hydroxybutyl, 3-hydroxybutyl 4-hydroxybutyl, 2,3-dihydroxypropyl, 1-(hydroxymethyl)-2-hydroxyethyl, 2,3-dihydroxybutyl, 3,4-dihydroxybutyl, and 2-(hydroxymethyl)-3-hydroxypropyl, and the like.

“Optional” or “optionally” means that the subsequently described event or circumstance may or may not occur, and that the description includes instances where the event or circumstance occurs and instances in which it does not. For example, the phrase “which group is optionally substituted with one to three halo atoms” or “optionally substituted aryl” means that the group referred to may or may not be substituted in order to fall within the scope of the invention, and that the description includes both substituted and unsubstituted moieties.

As used herein, a “targeting agent” comprises any molecule that has the capacity to bind to a cell surface of a target cell population, including a receptor associated with the cell surface, such as a peptide or protein growth factor, cytokine, tumor-specific antigen, hormone, transfer protein or antibody, a monoclonal antibody (“mAb”), a non-peptide; and wherein the targeting agent may be an intact molecule, an analog or a fragment thereof or a synthetic or a functional equivalent thereof; and may be genetically engineered. A targeting agent has the capacity to bind to a defined population of cells and may bind through a receptor, substrate, antigenic determinant, or other binding site on the target cell population. Specific examples of targeting agents include, but are not limited to, antibodies as defined below, growth factors such as nerve growth factor (NGF), epidermal growth factor (EGF), tumor growth factors TGF-α and TGF-β, vaccinia virus growth factor (VVGF), platelet-derived growth factor (PDGF), any protein or polypeptide growth factor that is a ligand for receptors or other binding sites concentrated on tumor cell plasma membranes or contained within such cells; a tumor-specific antigen such as α-fetoprotein that targets tumor cells such as human β-lymphoma and T-cell leukemia cells, a prostate specific antigen that will concentrate in prostate adenocarcinoma cells, a carcinoembryonic antigen (CEA), or a transfer carrier protein such as transferrin which binds to tumor cells such as T-cell leukemia cells; hormones, such as estradiol, neurotensin, melanocyte-stimulating hormone (α-MSH), follicle-stimulating hormone, lutenizing hormone, and human growth hormone; peptides, such as bombesin, gastrin-releasing peptide, RDG peptide, substance P, neuromedin-B, neuromedin-C, and metenkephalin or any peptide hormone that will target tumor tissue, such as insulin or insulin-like growth factor, glucagon, thyrotropin (TSH) or thyrotropin releasing hormone (TRP), somatostatin, calcitonin, lysine bradykinin, and the like. Other suitable targeting agents include serum proteins, fibrinolytic enzymes, and biological response modifiers, such as interleukin, interferon, erythropoietin, colony-stimulating factor, steroids, carbohydrates and lectins. Many of the targeting agents mentioned above are commercially available through Sigma Chemical Co., St. Louis, Mo., Calbiochem Co., La Jolla, Calif., and ICN Biomedical Co., Irvine, Calif., or can be isolated or synthesized by methods well known in the art, including recombinant DNA methods.

As used herein, “protein” refers to proteins, polypeptides, and peptides; and may be an intact molecule, a fragment thereof or a functional equivalent thereof, and may be genetically engineered; an example is an antibody, as defined below.

As used herein, an “antibody” encompasses polyclonal and monoclonal antibody preparations, as well as preparations including hybrid or chimeric antibodies, such as humanized antibodies, altered antibodies, F(ab′)₂ fragments, F(ab) fragments, Fv fragments, single domain antibodies, dimeric and trimeric antibody fragment constructs, minibodies, and functional fragments thereof which exhibit immunological binding properties of the parent antibody molecule and/or which bind a cell surface antigen.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments that exhibit immunological binding properties of the parent monoclonal antibody molecule.

As used herein, a “diagnostically or therapeutically effective agent” refers to an agent capable of exerting a diagnostic or a therapeutic effect when released from the bioconjugate. Such agents include diagnostic compounds such as, but not limited to, radioisotopes, radiopaque dyes, fluorogenic compounds, marker compounds, lectins and the like. Suitable therapeutic agents include, but are not limited to radioisotopes, cancer chemotherapeutic agents, toxins and other cytotoxic agents. In preferred embodiments, the radioisotopes are contained within carrier molecules which include, but are not limited to, an aryl glycoside, pyridinecarboxylate, and DOTA Examples of such agents include, but are not limited to, 5-iodo-3-pyridinecarboxylate; metal chelates wherein a macrocyclic carrier, such as 1,4,7,10-tetraazacyclododecane-N,N′,N′,N′″-tetraacetic acid (DOTA), forms a covalent complex with a radioisotope, such as Y-90 and the like.

The terms “radioisotope” and “radionuclide” are used interchangeably, and refer to an isotopic form of an element (either natural or artificial) that exhibits radioactivity. Artificial radioisotopes are made by neutron bombardment of stable isotopes in nuclear reactor. Preferred radioisotopes (radionuclides) for the radiodiagnostic and radiotherapeutic compounds include, but are not limited to, Cu-64, Ga-67, Ga-68, Zr-89, Ru-97, Tc-99m, Rh-105, Pd-109, In-111, I-123, I-125, I-131, Y-90, Re-186, Re-188, Au-198, Au-199, Pb-203, At-211, Pb-212 and Bi-212.

As used herein, the term “exogenous enzyme” is an enzyme that is not normally associated with the cells targeted by the bioconjugates of the invention, i.e. the enzyme is not normally present in, produced by, or found in association with the targeted cells. The exogenous enzyme is administered without an associated carrier or targeting moiety, such as an antibody or expression of the exogenous enzyme may be induced in the target cell by, for example, chemical or ligand induction. Examples of exogenous enzymes include, but are not limited to, β-lactamase, and the like.

As used herein, the term “β-lactamase” refers to any enzyme capable of hydrolyzing the CO—N bond of a β-lactam ring. These enzymes are available commercially, such as E. coli or B. cereus β-lactamases, or they may be cloned and expressed using recombinant DNA techniques well known in the art. The β-lactamases are reviewed in Bush, Antimicrobial. Agents Chemother., 33:259, 1989.

By “metabolizable linker moiety” is meant the portion of the bioconjugate composition that is capable of being cleaved by an exogenous enzyme as described above, such as, e.g. β-lactamase and the like.

By “β-lactamase sensitive linker” is meant a molecule that serves to link or conjugate a targeting agent, such as an antibody, to a diagnostically or a therapeutically effective agent, such as a radioisotope, which linker molecule is capable of being cleaved by β-lactamase.

As used herein, a “pharmaceutically acceptable vehicle” refers to a vehicle that is useful in preparing a pharmaceutical composition that is generally compatible with the other ingredients of the composition, not deleterious to the recipient, and neither biologically nor otherwise undesirable, and includes a vehicle that is acceptable for veterinary use as well as human pharmaceutical use. A “pharmaceutically acceptable vehicle” includes one and more than one such vehicles.

As used herein, a “pharmaceutically acceptable salt” of a compound refers to a salt that is pharmaceutically acceptable, as described above, and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, benzenesulfonic acid, benzoic acid, 3-(4-hydroxybenzoyl)benzoic acid, camphorsulfonic acid, p-chlorobenzenesulfonic acid, cinnamic acid, citric acid, cyclopentanepropionic acid, ethanesulfonic acid, 1,2-ethanedisulfonic acid, fumaric acid, glucoheptonic acid, gluconic acid, glutamic acid, glycolic acid, hexanoic acid, heptanoic acid, (o-hydroxybenzoyl) benzoic acid, hydroxynaphthoic acid, 2-hydroxyethanesulfonic acid, lactic acid, lauryl sulfuric acid, malic acid, maleic acid, malonic acid, mandelic acid, methanesulfonic acid, 4-methylbicyclo[2.2.2]oct-2-ene-1-carboxylic acid, 4,4′-methylenebis(3-hydroxy-2-ene-1-carboxylic acid), muconic acid, 2-naphthalenesulfonic acid, oxalic acid, 3-phenylpropionic acid, propionic acid, pyruvic acid, salicylic acid, stearic acid, succinic acid, tartaric acid, trimethylacetic acid, tertiary butylacetic acid, p-toluenesulfonic acid, and the like; or

(2) salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methyl-glucamine, triethanolamine, tromethamine, and the like. Acceptable inorganic bases include aluminum hydroxide, calcium hydroxide, potassium hydroxide, sodium carbonate, and sodium hydroxide. The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

As used herein, a “pharmaceutically acceptable hydrates” refers to hydrates, which are pharmaceutically acceptable, as defined above, and which possess the desired pharmacological activity. Such hydrates are formed by the combination of one or more molecules of water with one of the substances, in which the water retains its molecular state as H₂O, such combination being able to form one or more than one hydrate.

As used herein, a “therapeutically effective amount” refers to an amount of a compound that, when administered to a subject for treating a disease, is sufficient to effect such treatment for the disease as defined below. The “therapeutically effective amount” will vary depending on the diagnostically or therapeutically effective agent, disease state being treated, the severity of the disease treated, the age and relative health of the subject, the route and form of administration, the judgement of the attending medical or veterinary practitioner, and other factors.

As used herein, the term “pharmacological effect” encompasses effects produced in the subject that achieve the intended purpose of a therapy. In one preferred embodiment, a pharmacological effect means the targeted delivery of radiolabeled bioconjugate to the tumor tissue. For example, a pharmacological effect would be one that results in a greater retention of the radioisotope in tumor compared to normal tissue. Additionally, rapid removal of the circulating nonbound radioisotope from the system results in a reduction in the amount of radiation to normal organs, thus improving the delivery of radioisotope to tumor as compared to normal tissue.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms; or relieving the disease, i.e., causing regression of the disease or its clinical symptoms.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Preferred Compounds

The present invention provides bioconjugates and compositions comprising the same, for targeted delivery to selected cell populations. As explained above, the bioconjugates include a targeting agent, conjugated to a diagnostically or a therapeutically effective agent by a metabolizable linker moiety which is cleaved, e.g. in vivo, by an exogenous enzyme, delivered to the subject before, after or concurrently with the bioconjugate.

Bioconjugates of the invention may comprise the formula (I):

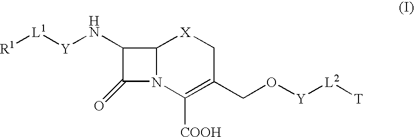

wherein m is an integer ranging from 1 to 12 inclusive; and n is an integer ranging from 1 to 12 inclusive;

L¹ is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —(CHR²)ₙ—CH₂—O—; —(CHR²)ₙ—; —NH—(CHR²)ₙ—NH—; —NH—(CHR²)ₙ—NH—CO—(CHR²)ₘ—CO-Z-; —(CHR²)ₙ—NH—CS—NH—(CHR²)ₘ—CS—NH-Z; —NH—(CHR²)ₙ—NH—CS—(CHR²)ₘ—CO-Z-; —(CHR²)ₙ—NH—CO—NH—(CHR²)ₘ—CO—NH-Z; or a biodegradable polyamino acid macromolecular carrier, wherein L¹-Y—NH taken together optionally form a heterocyclic or a heteroaryl ring;

L² is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —NH—(CHR²)ₙ—NH—; —NH—(CHR²)ₙ—(CHR³)—NH—; —NH—(CHR²)ₙ—NH—CO—(CHR²)ₘ—CO-Z-; —NH—(CHR²)ₙ—NH—CO—(CHR²)ₘ—CO—; —NH—(CHR²)ₙ—NH—CS—(CHR²)ₘ—CO-Z-; —NH—(CHR²)ₙ—NH—CS—(CHR²)ₘ—CO—; —(CHR²)ₙ—CH₂—O—; —(CHR²)ₙ—; or a biodegradable polyamino acid macromolecular carrier; wherein L² optionally forms cyclic structure comprising an aryl ring, heteroaryl ring, cycloalkyl ring, cycloalkenyl ring, wherein said ring is optionally substituted;

T is a targeting agent;

X is O, NH, S or SO;

Y is CO or CS;

Z is an amino acid, N-hydroxysuccinimydl (NHS) or sulfonated N-hydroxysuccinimydl;

R¹ is a diagnostically or therapeutically effective agent;

R² is H, OH, lower alkyl, alkoxy, acyloxy, alkylamino, alkylthio or hydroxyalkyl;

R³ is —COOH or —CH₂OSO₃H; or

a pharmaceutically acceptable salt thereof.

In another aspect, the invention relates to a bioconjugate composition comprising the formula (II):

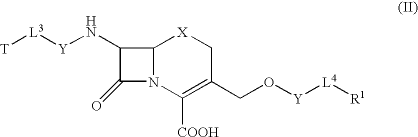

wherein m is an integer ranging from 1 to 12 inclusive; and n is an integer ranging from 1 to 12 inclusive;

L³ is —(CHR²)ₙ—NH—(CHR²)ₘ—CO-Z; —(CHR²)ₙ—NH—CO—(CHR²)ₘ—CO-Z; —(CHR²)ₙ—CO—NH-Z; —(CHR₂)ₙ—NH—; —(CHR²)ₙ—NH—CO—NH—(CHR²)ₘ—CO—NH-Z-; —(CHR²)ₙ—CH₂—S—; —(CHR²)ₙ—CH₂—O—; —NH—(CHR²)ₙ—NH—CS—(CHR²)ₘ—CO-Z; —NH—(CHR²)ₙ—NH—; —NH—(CHR²)ₙ—NH—CO—(CHR²)ₘ—CO-Z; —(CHR₂)ₙ—; —(CHR²)ₙ—NH—CS—NH—(CHR²)ₘ—CS—NH-Z; or a biodegradable polyamino acid macromolecular carrier, wherein L³-Y—NH taken together optionally form a heterocyclic or a heteroaryl ring;

L⁴ is —(CHR²)ₙ—NH—(CHR²)ₘ—CO-Z; CHR²)ₙ—NH—CO—(CHR²)ₘ—CO-Z; —(CHR²)ₙ—NH—; —(CHR²)ₙ—CH₂—S—; —NH—(CHR²)ₙ—NH—CO—(CHR²)ₘ—CO-Z-; —(CHR²)ₙ—CH₂—O—; —(CHR²)ₙ—; —NH—(CHR²)ₙ—NH—; —NH—(CHR)ⁿ—(R³)—NH—; —NH—(CHR²)ₙ—NH—CO—(CHR²)ₘ—CO—; —NH—(CHR³)ₙ—NH—CS—(CHR²)ₘ—CO-Z-; —NH—(CHR²)ₙ—NH—CS—(CHR²)ₘ—CO—; or a biodegradable polyamino acid macromolecular carrier, wherein L⁴ optionally forms cyclic structure comprising an aryl ring, heteroaryl ring, cycloalkyl ring, cycloalkenyl ring, wherein said ring is optionally substituted;

T is a targeting agent;

X is O, NH, S or SO;

Y is CO or CS;

Z is an amino acid, N-hydroxysuccinimydl (NHS) or sulfonated N-hydroxysuccinimydl;

R¹ is a diagnostically or therapeutically effective agent;

R² is H, OH, lower alkyl alkoxy, acyloxy, alkylamino, alkylthio or hydroxyalkyl;

R³ is —COOH or —CH₂OSO₃H; or

a pharmaceutically acceptable salt thereof.

In a preferred embodiment, the amino acid is selected from the group consisting of lysine, serine, threonine, tyrosine and cysteine; T is an antibody, more preferably an anti-CD19 antibody, an anti-CD20 antibody, an anti-CD22 antibody, an anti-CD33 antibody, an anti-CD37 antibody or an anti-CD45 antibody; and R¹ is a radioisotope, more preferably I-131, iodinated(I-131) aryl glycoside, 5-iodo(I-131)-3-pyridine-carboxylate, Y-90 within metal chelates.

Particularly preferred compounds of Formula (I), or a pharmaceutically acceptable salt or hydrate thereof, include:

bioconjugates of formula (I-A)

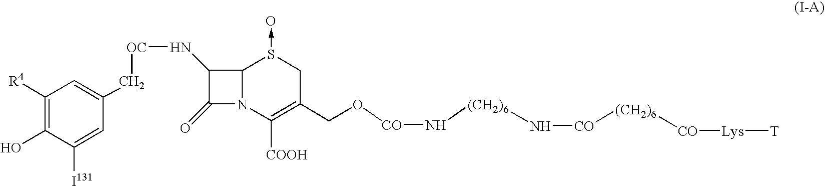

wherein T is an antibody, biotin, streptavidin or avidin; and R⁴ is H or I¹³¹.

Particularly preferred compounds of Formula (II), or a pharmaceutically acceptable salt or hydrate thereof include:

bioconjugates of formula (II-A)

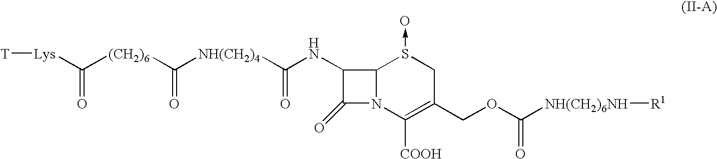

wherein T is an antibody, biotin, streptavidin or avidin; and

R¹ is an iodinated(I-131) aryl glycoside, 5-iodo(I-131)-3-pyridinecarboxyl or Y-90 1,4,7,10tetraazacyclododecane-N,N′,N″,N′″-tetraacetic acid (DOTA) complex;

bioconjugates of formula (II-B)

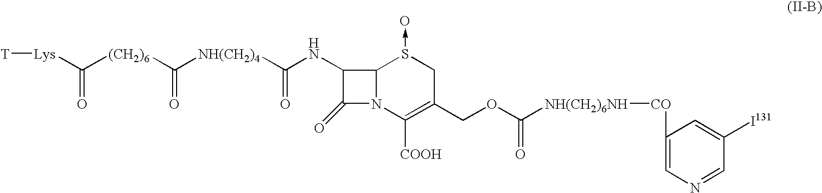

wherein T is an antibody, biotin, streptavidin or avidin;

bioconjugates of formula (II-C)

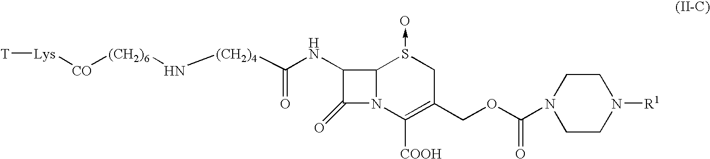

wherein T is an antibody, biotin, streptavidin or avidin, and

R¹ is an iodinated(I-131) aryl glycoside, 5-iodo(I-131)-3-pyridinecarboxyl or Y-90 1,4,7,10-tetraazacyclododecane-N,N′,N″,N′″-tetraacetic acid (DOTA) complex; and

bioconjugates of formula (II-D)

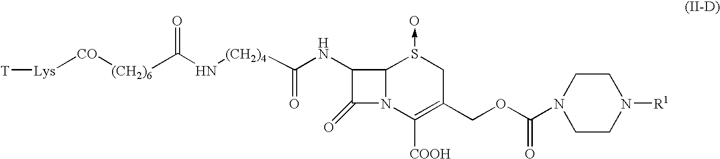

wherein T is an antibody, biotin, streptavidin or avidin; and R¹ is

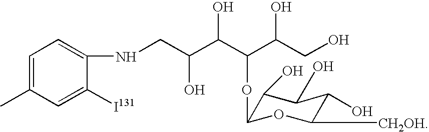

General Synthetic Schemes

Bioconjugates of this invention can be made by the methods depicted in the reaction schemes shown below.

The starting materials and reagents used in preparing these compounds are either available from commercial suppliers, such as Aldrich Chemical Co., or are prepared by methods known to those skilled in the art following procedures set forth in references such as Fieser and Fieser's Reagents for Organic Synthesis, Wiley & Sons, New York, 1991, Volumes 1-15; Rodd's Chemistry of Carbon Compounds, Elsevier Science Publishers, 1989, Volumes 1-5 and Supplementals; and Organic Reactions, Wiley & Sons, New York, 1991, Volumes 1-40. The following schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to, filtration, distillation, crystallization, chromatography, and the like. The reactions may be monitored using conventional techniques, including but not limited to, chromatography, e.g., analytical reverse phase chromatography (HPLC), and the like. Such materials may be characterized using conventional means, including physical constants and spectral data.

Unless specified to the contrary, the reactions described herein take place at atmospheric pressure over a temperature range from about −100° C. to about 250° C., more preferably from about −20° C. to about 125° C.

Bioconjugates of Formulas (I) and (II) are prepared using general methods described in the literature. In particular, bioconjugates of Formulae (I) and (II) (compounds 5A and 5B respectively) are generally prepared as set forth in reaction Scheme 1. An amino-carboxylic acid is treated with an appropriately activated protected acid (P¹-L-COOH) to yield compound 1 (for further details see, e.g., Examples 1-3, infra), wherein L is any one of L¹, L², L³ and L⁴ as defined above, and P¹ denotes suitable protecting groups for amino acids as described above (e.g., carbamates such as t-butoxycarbonyl (Boc), CBZ; amides such as benzoyl, allyl; and acetals such as methoxymethyl). Compound 1 is hydrolyzed using, e.g., sodium hydroxide in aqueous methanol. The resulting compound is treated with an appropriately activated protecting agent P², wherein P² denotes suitable protecting group as described above (e.g., diphenylmethyl, p-methoxybenzyl, alkyl, allyl and trialkylsilyl) to yield the ester 2 (for further details see, e.g., Examples 1-3, infra). Compound 2 is acylated with an appropriately activated acylating agent, (e.g. a phosgene derivative such as Cl—CO—OCCl₃), to yield an intermediate 3. The intermediate 3 is optionally oxidized (wherein X═SO) using, e.g., mCPBA in dichloromethane (0° C., for about 10 min to about 40 min).

Acylation of compound 2 can be carried out in a suitable solvent (e.g., DMSO, THF, and the like), with a suitable base present (diisopropyl ethyl amine (DIEA), triethyl amine (TEA) and the like) at about −40° C. to about 250° C., typically at about −30° C. to about 150° C. and preferably at about −20° C. to about 100° C., requiring about 1 min to about 72 hours, preferably about 1 min to about 60 min. more preferably about 5 min to about 20 min. Deprotection can be effected by any means which remove the protective group and give the desired product As described above, a detailed description of the techniques applicable to protective groups and their removal can be found in T. W. Greene, Protective Groups in Organic Synthesis, Wiley and Sons, New York, 1991. For example, a convenient method of deprotection when the protective group is tert-butoxycarbonyl can be carried out with trifluoroacetic acid (TFA) or hydrochloric acid in a suitable inert organic (e.g., ethyl acetate, dichloromethane, tetrahydrofuran (THF), hexamethylphosphoramide (HMPA), or any appropriate mixture of suitable solvents, etc., preferably THF, ethyl acetate or TFA/anisole) at about 0° C. to about 250° C., typically at about 10° C. to about 100° C. and preferably at about 20° C. to about 40° C., requiring about 1 min to about 72 hours, preferably about 1 min to about 60 min, more preferably about 5 min to about 40 min (for further details see, e.g., Example 1, infra). Deprotection, when the protective group is benzyl, can be carried out by catalytic hydrogenation The hydrogenation is carried out with a suitable catalyst (e.g., 10% palladium on carbon (10% Pd/C), palladium hydroxide, palladium acetate, etc. preferably 10% Pd/C) in the presence of ammonium formate and in an appropriate solvent, typically an alcohol (e.g., ethanol, methanol, isopropanol, any appropriate mixture of alcohols, etc.), preferably methanol, at about 0° to about 250° C., typically at about 10° to about 150° C. and preferably at about 20° to about 100° C. and preferably at reflux. Alternatively, the benzyl group can be removed by treating the protected compound with the catalyst under a hydrogen atmosphere at 0 to 50 psi, typically at 10 to 20 psi and preferably at approximately 15 psi, at about 0° to about 250° C., typically at about 100 to about 150° C. and preferably at about 20° to about 100° C., requiring about 1 min to about 72 hours, preferably about 1 min to about 60 min, more preferably about 5 min to about 40 min.

The intermediate 3 is treated with an appropriately activated protected amine (P³—L′—NH₂), to yield compound 4 (for further details see, e.g., Examples 1-3, infra), wherein L′ is any one of L¹, L², L³ and L⁴ as defined above, and P³ is a suitable protecting groups for amino acids as described above. Compound 4 is deprotected and the resulting amine is conjugated with an appropriate linker, e.g. a NHS-linker. The reaction can be carried out in a suitable solvent (e.g. DMSO) with a suitable base present (e.g., DIEA, chloramine-T) at about 0° C. to about 250° C., typically at about 10° C. to about 150° C. and preferably at about 20° C. to about 40° C., requiring about 1 min to about 72 hours, preferably about 1 min to about 60 min, more preferably about 5 min to about 20 min.

The resulting compound is treated with an appropriately activated diagnostically or therapeutically effective agent (R¹), wherein R¹ is as defined above, e.g. chloramine-T/NaI¹³¹ or iodogen beads/I¹³¹ (for further details see, e.g., Examples 1-3, infra). The reaction can be carried out in a suitable aqueous solvent at about 0° C. to about 250° C., typically at about 5° C. to about 100° C. and preferably at about 10° C. to about 40° C., requiring about 1 min to about 72 hours, preferably about 1 min to about 60 min, more preferably about 5 min to about 20 min.

The resulting derivative is incubated with an appropriately activated targeting agent (T), wherein T is as defined above (e.g., an amino acid-antibody conjugate wherein the amino acid is preferably selected from the group consisting of lysine, serine, threonine, tyrosine and cysteine; more preferably a lysine-antibody conjugate), to yield the corresponding bioconjugates 5A or 5B, corresponding to Formulae (I) and (II) respectively, (for further details see, e.g., Examples 1-3, infra). The reaction can be carried out in a suitable solvent (e.g., an aqueous borate buffer) at a pH of about 5 to about 9, preferably about 6 to 8, more preferably about 7 to about 8, at about 0° C. to about 250° C., typically at about 5° C. to about 100° C. and preferably at about 20° C. to about 40° C., requiring about 1 min to about 72 hours, preferably about 5 min to about 60 min, more preferably about 10 min to about 40 min.

Particularly preferred targeting agents are antibodies directed against cell surface proteins which are present on the targeted cells. The antibodies are prepared as described further below. The antibodies are bound to the diagnostic and therapeutic agents described above to form the bioconjugates of the invention, using techniques well established in the art. The antibodies may be covalently or non-covalently associated with the diagnostic and therapeutic agents.

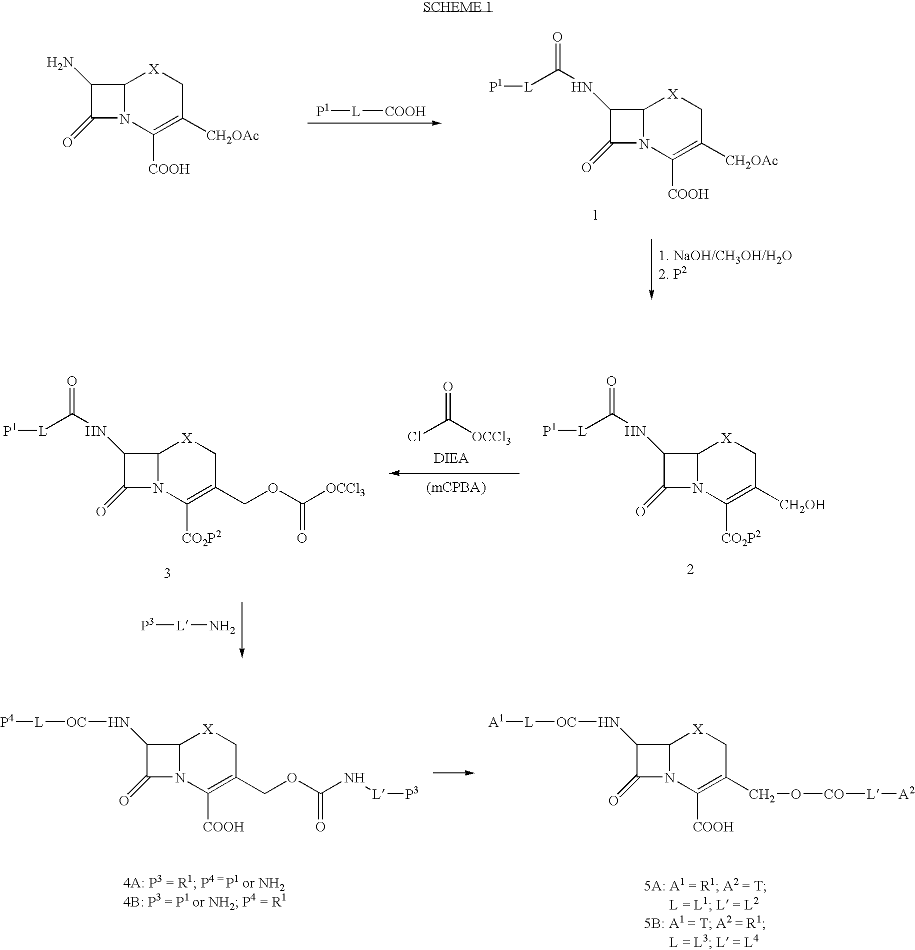

Particularly preferred diagnostically effective agents include diagnostic compounds such as, but not limited to, radioisotopes, radiopaque dyes, fluorogenic compounds, marker compounds, lectins and the like. Suitable therapeutic agents include, but are not limited to radioisotopes, cancer chemotherapeutic agents, toxins and other cytotoxic agents. Preferred radioisotopes include, but are not limited to, Cu-64, Ga-67, Ga-68, Zr-89, Ru-97, Tc-99m, Rh-105, Pd-109, In-111, I-123, I-125, I-131, Y-90, Re-186, Re-188, Au-198, Au-199, Pb-203, At-211, Pb-212 and Bi-212. In preferred embodiments, the radioisotopes are contained within carrier molecules which include, but are not limited to, an aryl glycoside, pyridinecarboxylate, and DOTA. Examples of such agents include, but are not limited to, 5-iodo-3-pyridinecarboxylate; metal chelates wherein a macrocyclic carrier, such as 1,4,7,10-tetraazacyclododecane-N,N′,N″,N′″-tetraacetic acid (DOTA), forms a covalent complex with a radioisotope, such as Y-90 and the like. Aryl glycosides are prepared as described further below (see Scheme 2).

In particular, aryl glycosides are generally prepared as set forth in reaction Scheme 2. An amino-carboxylic acid 31 is treated with an appropriately activated protected amine 32 to yield compound 33 (for further details see, e.g., Example 3, infra), wherein Ar denotes aryl or heteroaryl as defined above (e.g., phenyl naphthyl, furanyl, pyrrole, pyridine, and the like), L and L′ are as defined above, and P denotes suitable protecting group as described above. Compound 33 is treated with an appropriately activated sugar (e.g., cellobiose, glucose, lactose) in a suitable solvent (e.g., H₂O:EtOH—70:30, and the like), with a suitable base present (e.g. NaCNBH₃), at a pH of about 3.5 to about 9, preferably about 4 to 7, more preferably about 4.5 to about 5.5, at about 0° C. to about 250° C., typically at about 20° C. to about 150° C. and preferably at about 75° C. to about 120° C., requiring about 60 min to about 168 hours, preferably about 24 h to about 144 h, more preferably about 48 h to about 120 h, to yield compound 34 (for further details see, e.g., Example 3, infra). Compound 34 is deprotected as described above to yield the aryl glycoside 35.

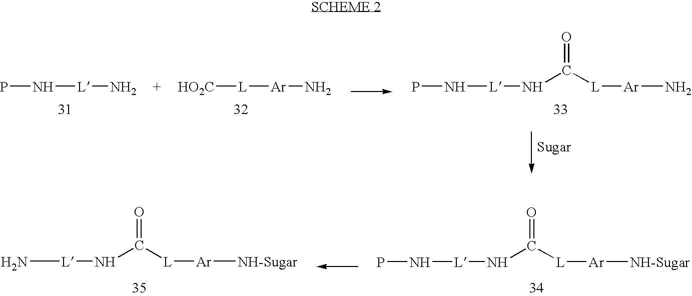

Preparation of Antibodies

As explained above, the present invention encompasses bioconjugates that include targeting agents for targeting specific cell populations. Particularly preferred targeting agents are antibodies directed against cell surface proteins which are present on the targeted cells. Antibodies that will find use with the present bioconjugates include conventional polyclonal and monoclonal antibodies, as well as hybrid or chimeric antibodies such as humanized antibodies, altered antibodies, antibody fragments such as F(ab) fragments, F(ab′)₂ fragments, Fv fragments, single domain antibodies, dimeric and trimeric antibody fragments, minibodies, and the like.

For purposes of the following discussion, the “antigen-binding site,” or “binding portion” of an antibody refers to the part of the immunoglobulin molecule that participates in antigen binding. The antigen binding site is typically formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “R” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies for use with the present invention can be produced using techniques well established in the art. For example, polyclonal antibodies are generated by immunizing a suitable animal, such as a mouse, rat, rabbit, sheep or goat, with an antigen of interest. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid. aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof Rabbits, sheep and goats are preferred for the preparation of polyclonal sera when large volumes of sera are desired. These animals are good design choices also because of the availability of labeled anti-rabbit, anti-sheep and anti-goat antibodies. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art Polyclonal antisera is then obtained from the immunized animal.

Monoclonal antibodies are generally prepared using the method of Kohler and Milstein, Nature (1975) 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Monoclonal antibodies or portions thereof may be identified by first screening a B-cell cDNA library for DNA molecule that encode antibodies that specifically bind to the cell surface protein of interest e.g. CD91, CD20, CD22 and the like, according to the method generally set forth by Huse et al., (Science 246:1275-1281, 1989, incorporated by reference herein in its entirety). The DNA molecule may then be cloned and amplified to obtain sequences that encode the antibody (or binding domain) of the desired specificity.

As explained above, antibody fragments which retain the ability to recognize the targeted cell, will also find use in the subject bioconjugates. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as Fᵥ. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv” or “scFv”) polypeptide is a covalently linked V_{H}-V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al. (1988) Proc. Nat. Acad. Sci. USA 85(16):5879-5883; U.S. Pat. Nos. 5,091,513, 5,132,405 and 4,946,778. Design criteria include determining the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

One method of obtaining nucleotide sequences encoding sFv molecules is by an overlap PCR approach. See, e.g., Horton et al. (1990) BioTechniques 8:528-535. The ends of the light and heavy chain variable regions that are to be joined through a linker sequence are first extended by PCR amplification of each variable region, using primers that contain the terminal sequence of the variable region followed by all or most of the desired linker sequence. After this extension step, the light and heavy chain variable regions contain overlapping extensions which jointly contain the entire linker sequence, and which can be annealed at the overlap and extended by PCR to obtain the complete sFv sequence using methods known in the art.

“Mini-antibodies“or “minibodies” will also find use with the present invention. Minibodies are sFv polypeptide chains which include oligomerization domains at their C-termini, separated from the sFv by a hinge region. Pack et al, (1992), Biochem, 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein.

Generally, minibodies are produced using recombinant methods well known in the art. See, e.g., Pack et al, (1992), Biochem, 31:1579-1584; Cumber et al., 1992, J. Immunology, 149B:120-126; and International application Nos. PCT/US92/07986, published Apr. 1, 1993, and PCT/US92/10140, published Jun. 10, 1993, as well as examples 6 and 8, below. For example, International application PCT/US92/07986 describes methods for making bifunctional F(ab′)₂ molecules composed of two F(ab′) monomers linked through cysteine amino acids located at the C-terminus of the first constant domain of each heavy chain. International application PCT/US92/10140 also discloses bifunctional F(ab′)₂ dimers which, in addition to the cysteine residues located in the hinge region, also contain C-terminal leucine zipper domains that further stabilize the F(ab′)₂ dimers. In both cases, the resulting F(ab′)₂ dimers are ≧100 kD in size, and thus smaller than intact immunoglobulins. The generation of (FvCys)₂ heterodimers by chemically crosslinking two V_{H}.CYS domains together is described by Cumber et al., 1992, J. Immunology, 149B:120-126.

Chimeric antibody molecules will also find use with the present invention. A chimeric antibody can include antigen-binding sites, such as variable regions, or fragments of variable regions, derived from a non-human immunoglobulin, which retain specificity for the cell-surface receptor or antigen in question. The remainder of the antibody can be derived from the species in which the antibody will be used. Thus, if the antibody is to be used in a human, the antibody can be “humanized” in order to reduce immunogenicity yet retain activity. Such chimeric antibodies may contain not only combining sites for the cell-surface receptor or antigen of interest, but also binding sites for other proteins. In this way, bifunctional reagents can be generated with targeted specificity to, e.g., both external and internal antigens. For a description of chimeric antibodies and methods of generating the same, see, e.g., Winter et al. (1991) Nature 349:293-299; Lobuglio et al. (1989) Proc. Nat. Acad. Sci. USA 86:4220-4224; Shaw et al. (1987) J. Immunol. 138:4534-4538; and Brown et al. (1987) Cancer Res. 47:3577-3583) (each describing chimeric antibodies comprising rodent V regions and associated CDRs fused to human constant domains); Jones et al. (1986) Nature 321:522-525; Riechmann et al. (1988) 332:323-327; and Verhoeyen et al. (1988) Science 239:1534-1536 (each describing rodent CDRs grafted into a human supporting FR prior to fusion with an appropriate human antibody constant domain); European Patent Publication No. 519,596, published Dec. 23, 1992 (describing rodent CDRs supported by recombinantly veneered rodent FRs).

Antibodies with veneered FRs can be produced as follows. Initially, the FR sequences derived from the V_{H} and V_{L} domains of an antibody molecule produced by hybridoma cell lines are compared with corresponding FR sequences of human variable domains obtained from an appropriate database. See, e.g., Kabat et al., in Sequences of Proteins of Immunological Interest, 4th ed., (U.S. Dept. of Health and Human Services, U.S. Government Printing Office, 1987) and updates to the database. Human frameworks with a high degree of sequence similarity to those of the murine regions are identified. Sequence similarity is measured using identical residues as well as evolutionarily conservative amino acid substitutions. Similarity searches are performed using the selected murine framework sequence from which the CDRs have been removed. The framework sequence is used to query a database of human immunoglobulin sequences derived from multiple sources. Sequences with a high degree of sequence similarity are examined individually for their potential as humanizing framework sequences. In this way, the human homologue providing the CDRs from selected molecules with the structure most similar to their native murine framework is selected as the template for the construction of the veneered FRs.

The selected human V regions are then compared residue by residue to the corresponding murine amino acids. The residues in the murine FRs which differ from the selected human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” FRs are designed to retain the murine CDR residues, the residues substantially adjacent to the CDRs, the residues identified as buried or mostly buried (solvent inaccessible), the residues believed to participate in non-covalent (e.g., electrostatic) interchain contacts, and the residues from conserved structural regions of the FRs which are believed to influence the “canonical” tertiary structures of the CDR loops. Expression vectors including the recombinant nucleotide sequences encoding these molecules can be introduced into suitable host cells for the expression of recombinant human antibodies which exhibit the antigen specificity of the murine antibody molecule. Additionally, coexpression of complementary V_{H} and V_{L} molecules having veneered frameworks provides a convenient method of producing a heterodimeric polypeptide, featuring an antigen-binding site that binds specifically to, e.g., a human tumor antigen, and which is weakly-immunogenic, or substantially non-immunogenic in a human recipient. For a further description of the veneering process see, e.g., European Patent Publication No. 519,596 and International Publication No. WO 92/22653.

Examples of antibodies useful in the present invention include, but are not limited to, those which bind specifically to antigens found on carcinomas, melanomas, lymphomas, and bone and soft tissue sarcomas as well as other tumors. The antibodies used to practice the invention may be either internalizing (e.g., anti-CD20 antibodies) or non-internalizing antibodies (e.g., anti-CD19 or anti-CD22 antibodies).

Specific antibodies which may be used to deliver the diagnostically or therapeutically effective agent to the tumor site include, but are not limited to, L6, an IgG2a monoclonal antibody (hybridoma deposit no. ATCC HB8677) that binds to a glycoprotein antigen on human lung carcinoma cells (Hellstrom, et al., Proc. Natl. Acad. Sci. U.S.A., 83:7059, 1986); 96.5, an IgG2a monoclonal antibody that is specific for p97, a melanoma-associated antigen (Brown, et al., J. Immunol., 127:539, 1981); anti-CD20 antibodies such as B1 (L. M. Nadler, Leukocyte Typing II Vol. 2, Reinherz et al., eds., New York, Springer-Verlag, 1986; BioGenex Lab., San Ramon, Calif.) and 1F5, an IgG2a monoclonal antibody (hybridoma deposit no. ATCC HB9645) that is specific for the CD20 antigen on normal and neoplastic B cells (Clark et al., Proc. Natl. Acad. Sci. U.S.A, 82:1766, 1985); anti-CD19 antibodies such as B4 (L. M. Nadler, Leukocyte Typing II, 1986; BioGenex Lab., San Ramon, Calif.) and HD37 (Leukocyte Typing II, Vol. 2, pages 391-402, Reinherz et al., eds., New York, Springer-Verlag, 1986; Biomeda Corp, Foster City, Calif.); anti-CD22 antibodies such as HD39 (Roche Molecular Biomedicals, Palo Alto, Calif.) and 4KB128 (Moldenhauer et al., Leukocyte Typing II, Vol. 2, Reinherz et al., eds., New York, Springer-Verlag, 1986; Biomeda Corp., Foster City, Calif.); anti-CD37 antibodies (e.g., MB-1), and anti-CD45 antibodies (Leukocyte Typing II, 1986; hybridoma deposit no. ATCC BB 10508).

Once the antibodies are produced, they are bound to the diagnostic and therapeutic agents described above to form the bioconjugates of the invention, using techniques described above.

Administration and Pharmaceutical Compositions

The invention provides pharmaceutical compositions comprising a radioactive/therapeutic agent of the present invention or a pharmaceutically acceptable salt, hydrate or derivative thereof together with one or more pharmaceutically acceptable carriers, and optionally other therapeutic and/or prophylactic ingredients.

The bioconjugates of the invention can be administered as described below. The exogenous enzyme can be administered to the subject before, after or concurrently with the bioconjugate. Further, the bioconjugate and exogenous enzyme may be administered in vivo or in vitro, depending on the intended use. For example, for therapeutic purposes, the bioconjugate and enzyme are generally administered directly to the subject For diagnostic purposes, it may be desirable to administer the bioconjugate and enzyme in vitro, e.g., to biological samples derived from the subject, such as cells, blood, saliva, etc. Alternatively, diagnosis may also be carried out in vivo.

The exogenous enzyme will be administered in an amount effective to cleave the metabolizable linker moiety of the bioconjugate. Generally, the amount of enzyme delivered will depend upon the particular bioconjugate and enzyme in question. One of ordinary skill in the art will be able, without undue experimentation and in reliance upon personal knowledge and the disclosure of this application, to ascertain a therapeutically or diagnostically effective amount of the enzyme for use in diagnostic or therapeutic purposes.

The bioconjugates of this invention will be administered in a therapeutically or diagnostically effective amount by any of the accepted modes of administration for agents that serve similar utilities. Suitable dosage ranges are about 1 mg to about 500 mg, preferably about 1 mg to about 100 mg, and more preferably about 1 mg to about 30 mg, depending upon numerous factors such as the severity of the disease to be treated, the age and relative health of the subject, the potency of the compound used, the route and form of administration, the indication towards which the administration is directed, and the preferences and experience of the medical or veterinary practitioner involved One of ordinary skill in the art will be able, without undue experimentation and in reliance upon personal knowledge and the disclosure of this application, to ascertain a therapeutically or diagnostically effective amount of the compounds of this invention for use in treating a given disease.

In general, bioconjugates of this invention will be administered as pharmaceutical formulations including those suitable for oral (including buccal and sub-lingual), rectal, nasal, topical, pulmonary, vaginal or parenteral (including intramuscular, intraarterial, intrathecal, subcutaneous, and intravenous) administration or in a form suitable for administration by inhalation or insufflation.

The bioconjugates of the invention, together with a conventional adjuvant, vehicle, or diluent, may be placed into the form of pharmaceutical compositions and unit dosages. The pharmaceutical compositions and unit dosage forms may comprise conventional ingredients in conventional proportions, with or without additional active compounds or principles, and the unit dosage forms may contain any suitable effective amount of the active ingredient commensurate with the intended daily dosage range to be employed. The pharmaceutical composition may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use.

For example, the bioconjugates of the present invention may be formulated in a wide variety of administration dosage forms. The pharmaceutical compositions and dosage forms may comprise the compounds of the invention or its pharmaceutically acceptable salt or hydrate as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the finely divided active component In tablets, the active component is mixed with the carrier having the necessary binding capacity in suitable proportions and compacted in the shape and size desired. The powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations such as emulsions, syrups, elixirs, aqueous solutions, aqueous suspensions, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The bioconjugates of the present invention may be formulated for parenteral administration (e.g., by injection, for example bolus injection or continuous infusion) and may be presented in unit dose form in ampules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The bioconjugates of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The bioconjugates of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into conveniently sized molds, allowed to cool, and to solidify.

The bioconjugates of the present invention may be formulated for vaginal administration. Pessaries, tampons, creams, gels, pastes, foams or sprays, may contain agents in addition to the active ingredient, such carriers, known in the art to be appropriate.

The bioconjugates of the present invention may also be formulated for nasal administration. The solutions or suspensions are applied directly to the nasal cavity by conventional means, for example with a dropper, pipette or spray. The formulations may be provided in a single or multidose form. In the case of a dropper or pipette, this may be achieved by the patient administering an appropriate, predetermined volume of the solution or suspension. In the case of a spray, this may be achieved for example by means of a metering atomizing spray pump.

The bioconjugates of the present invention may also be formulated for aerosol administration, particularly to the respiratory tract including intranasal administration The bioconjugates will generally have a small particle size for example of the order of about 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafuoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of, e.g., gelatin or blister packs from which the powder may be administered by means of an inhaler.

When desired, formulations can be prepared with enteric coatings adapted for sustained or controlled release administration of the active ingredient.

Other suitable pharmaceutical carriers and their formulations are described in, e.g., Remington: The Science and Practice of Pharmacy 1995, edited by E. W. Martin, Mack Publishing Company, 19th edition, Easton, Pa.

Pharmacology and Utility

In a preferred embodiment, the bioconjugates of this invention are useful for treating disease indications, ameliorated by delivery of a diagnostic or a therapeutically effective agent to cells comprising administering a pharmaceutically effective amount of a bioconjugate as described above, wherein the targeting agent is reactive with a binding site on the surface of said cells; and administering a pharmaceutically effective amount of an exogenous enzyme capable of cleaving the metabolizable linkage. In more preferred embodiments, the cells are cancer cells, such as tumor cells.

In an alternative embodiment, the bioconjugates of the invention are useful for detecting the presence of a disease in a mammal suspected of having said disease, comprising administering to the mammal a diagnostically effective amount of a bioconjugate as described above, and an effective amount of an exogenous enzyme capable of cleaving the metabolizable linkage.

Assays:

The pharmacology of the bioconjugates of this invention was determined by art-recognized procedures. In vitro techniques for determining the β-lactamase sensitivity of the bioconjugates of the invention are described in Examples 4 and 5; and in vivo techniques for biodistribution and metabolism of the bioconjugates are described in Examples 9-16.

### EXAMPLES

The following preparations and examples are given to enable those skilled in the art to more clearly understand and practice the present invention. They should not be considered as limiting the scope of the invention, but merely as being illustrative and representative thereof

### Example 1

Synthesis of Bioconjugates of Formula I

Compound 14 was synthesized as illustrated in Scheme 3. Particularly, 7-amino-cephalosporic acetate (Aldrich Chemical Co.) was treated with t-butoxycarbonyl (Boc)-protected hydroxyphenylacetic acid in DCC in NMP (room temperature, 1 h). The resultant compound was treated with sodium hydroxide (2M) in methanol (−20° C., 5 min) to yield the compound 11. Compound 11 was treated with diphenylmethyl azide in methanol (0° C., 5 min) to yield the ester 12. The ester 12 was acylated with diisocyanohexane in DMSO (room temperature, 1 h) and hydrolyzed (aqueous acetic acid), followed by oxidization with mCPBA in methylene chloride (0° C., 20 min) to yield compound 13. Compound 13 was deprotected (50% TFA/anisole, 10 min) and the resulting amine was conjugated with an NHS-linker (trace DIEA/DMSO, room temperature, 30 min) to yield compound 14. The compounds 11-14 were fully characterized by proton NMR and mass spectroscopy (MS).

The conditions for iodination of compound 14, its conjugation, and subsequent susceptibility to cleavage by β-lactamase were evaluated in vitro by analytical HPLC and MS. Compound 14 was iodinated using cold NaI (2 equivalents) and an excess of chloramine-T. After approximately 1 minute, the reaction mixture was quenched with aqueous Na₂S₂O₃. The mixture was injected into a reverse phase HPLC column and eluted with an acetonitrile-water gradient to yield the corresponding diiodo derivative of 14 as a single compound

The diiodo derivative was incubated in an aqueous borate buffer (pH 7.8, 30 min) with a lysine-antibody conjugate to yield compound 15 (Formula I-A).

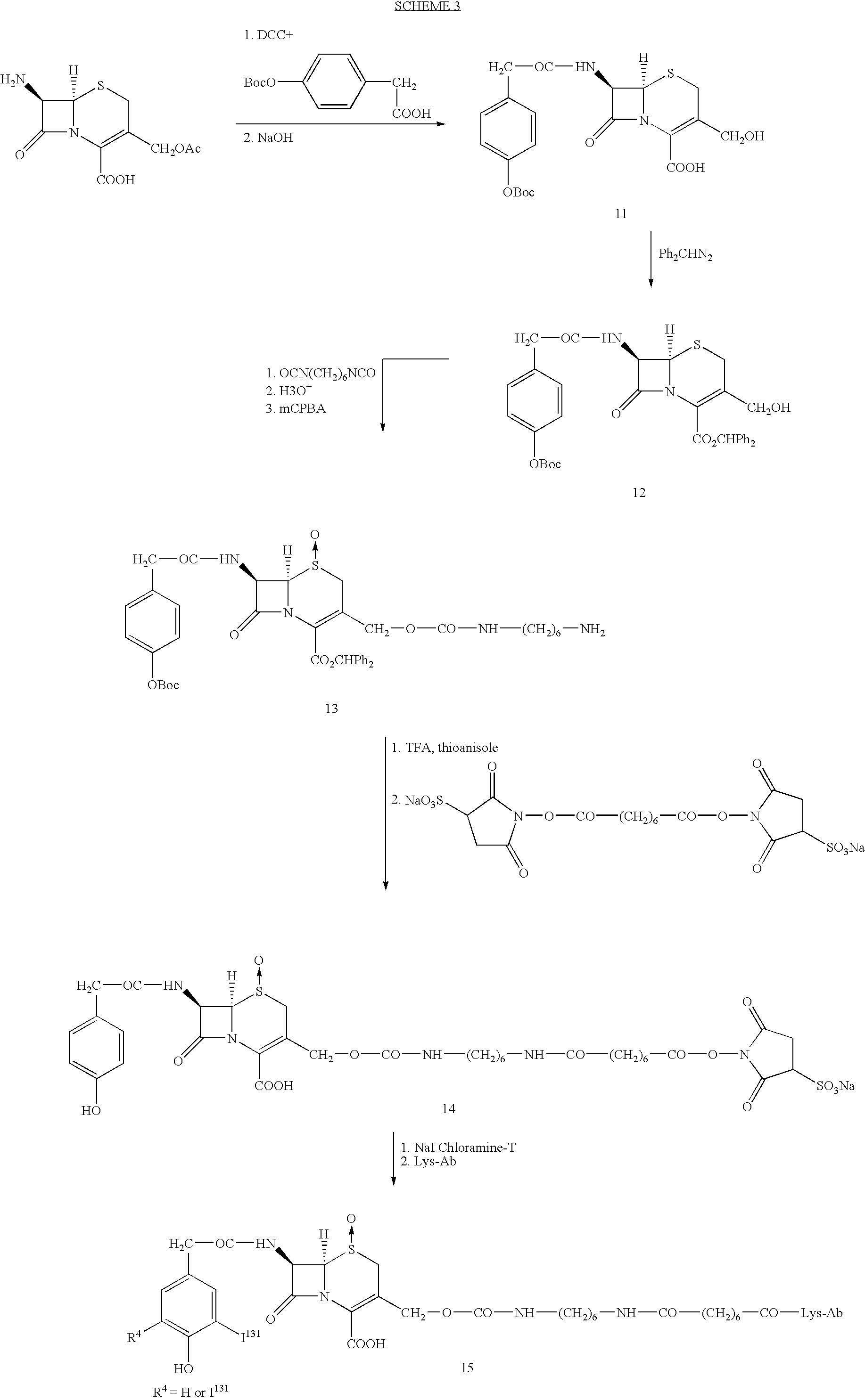

### Example 2

Synthesis of Bioconjugates of Formula II

Compound 21 is synthesized as illustrated in Scheme 4. Particularly, 7-amino-cephalosporic acetate (Aldrich Chemical Co.) was treated with t-butoxycarbonyl (Boc)-protected 1-aminopentanoic acid in DCC in NMP (room temperature, 1 h) to yield compound 16. Compound 16 was treated with sodium hydroxide (2M) in methanol (−20° C., 5 min), and the resulting compound was treated with diphenylmethyl azide in methanol (0° C., 30 min) to yield the ester 17. The ester 12 was acylated with a phosgene derivative and DIEA (THF, −20° C., 5 min), followed by oxidization with mCPBA in methylene chloride (0° C., 20 min) to yield compound 18. Compound 18 was treated with compound 24 (synthesized as described below in Scheme 5) in DMSO (room temperature, 30 min) to yield compound 19. Compound 19 was deprotected with 50% TFA/anisole (room temperature,10 min) and the resulting amine was conjugated with an NHS-linker (Pierce Corp.) (H₂O, room temperature, 30 min) to yield compound 20. Compound 20 was iodinated (I¹³¹) using chloramine T/NaI¹³¹ or Iodogen beads/NaI¹³¹ H₂O, 5 min). The iodo derivative was incubated in an aqueous borate buffer (pH 7.8, 30 min) with a lysine-antibody conjugate to yield compound 21 (Formula II).

Compound 24 was synthesized as described below in Scheme 5. In particular, compound 22 treated with n-butyl lithium/ethanol (−100° C., 10 min), followed by treatment with trimethyl tin chloride (−100° C., 10 min). The reaction mixture was warmed to 0° C. (30 min), followed by treatment with N,N′-disuccinimidyl carbonate (THF, room temperature, 1 h) to yield compound 23. Compound 23 was treated with 1,4-diaminobutane (acetonitrile, DIEA, room temperature) to yield compound 24.

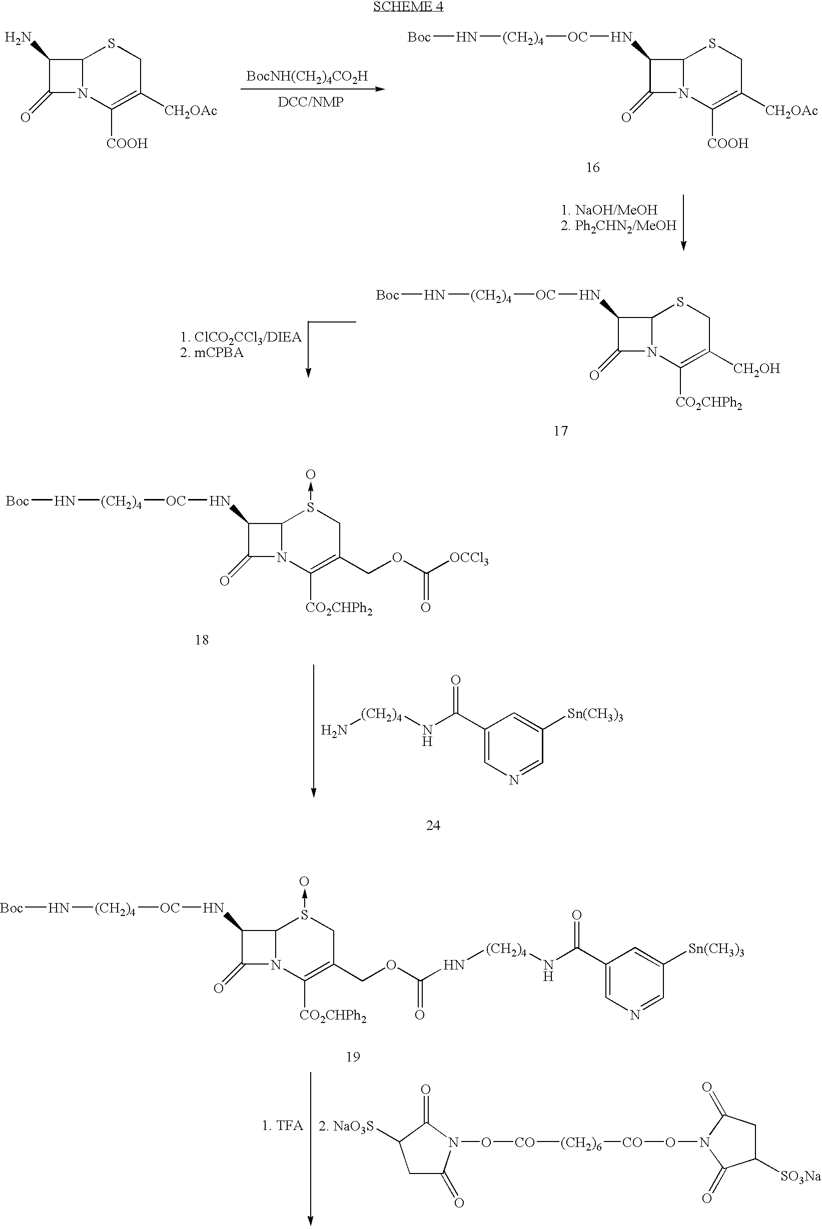

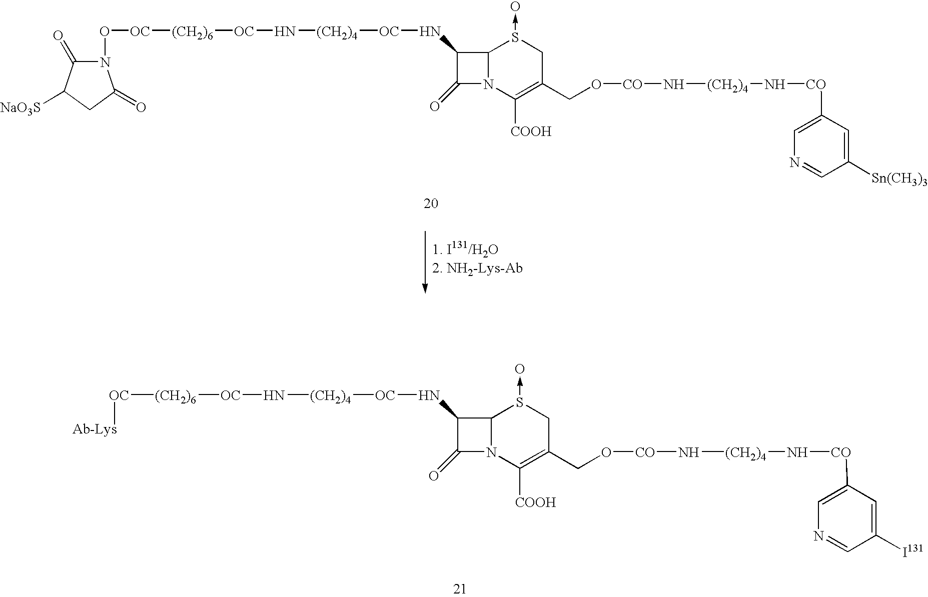

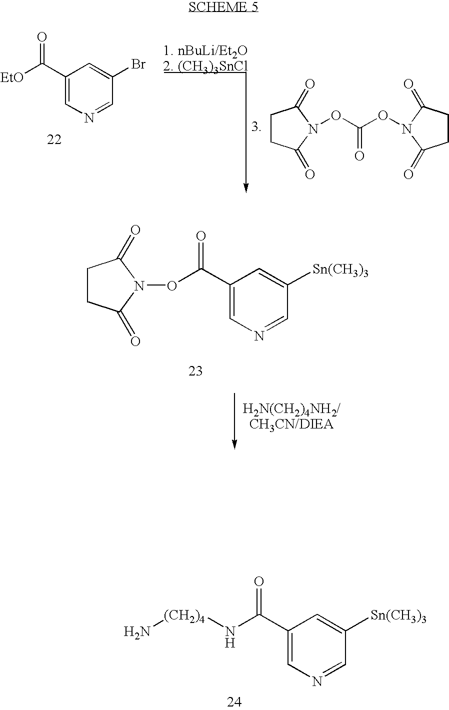

### Example 3

Synthesis of Bioconjugates of Formula II

Compound 27 is synthesized as illustrated in Scheme 6. Particularly, compound 18 was synthesized as described in Example 2 above. Compound 18 was treated with compound 30 (synthesized as described below in Scheme 7) in DMSO (room temperature, 30 min) to yield compound 25. Compound 25 was deprotected with 50% TFA/anisole (room temperature,10 min) and the resulting amine was conjugated with an NHS-linker (H₂O, room temperature, 30 min) to yield compound 26. Compound 20 was iodinated (I¹³¹) using chloramine T/NaI¹³¹ or Iodogen beads/NaI¹³¹ (H₂O, 5 min). The iodo derivative was incubated in an aqueous borate buffer (pH 7.8, 30 min) with a lysine-antibody conjugate to yield compound 27 (Formula II).

Compound 30 was synthesized as described below in Scheme 7. In particular, 4-aminobenzoic acid was treated with Boc-protected piperazine (DCC/DMF, room temperature, 2 h) to yield compound 28. Compound 28 was treated with cellobiose in the presence of NaBH₃CN (H₂O:EtOH—70:30; 90° C., 4 days) to yield compound 29. Compound 29 was deprotected (TFA:anisole 50:50; room temperature, 20 min) to yield compound 30.

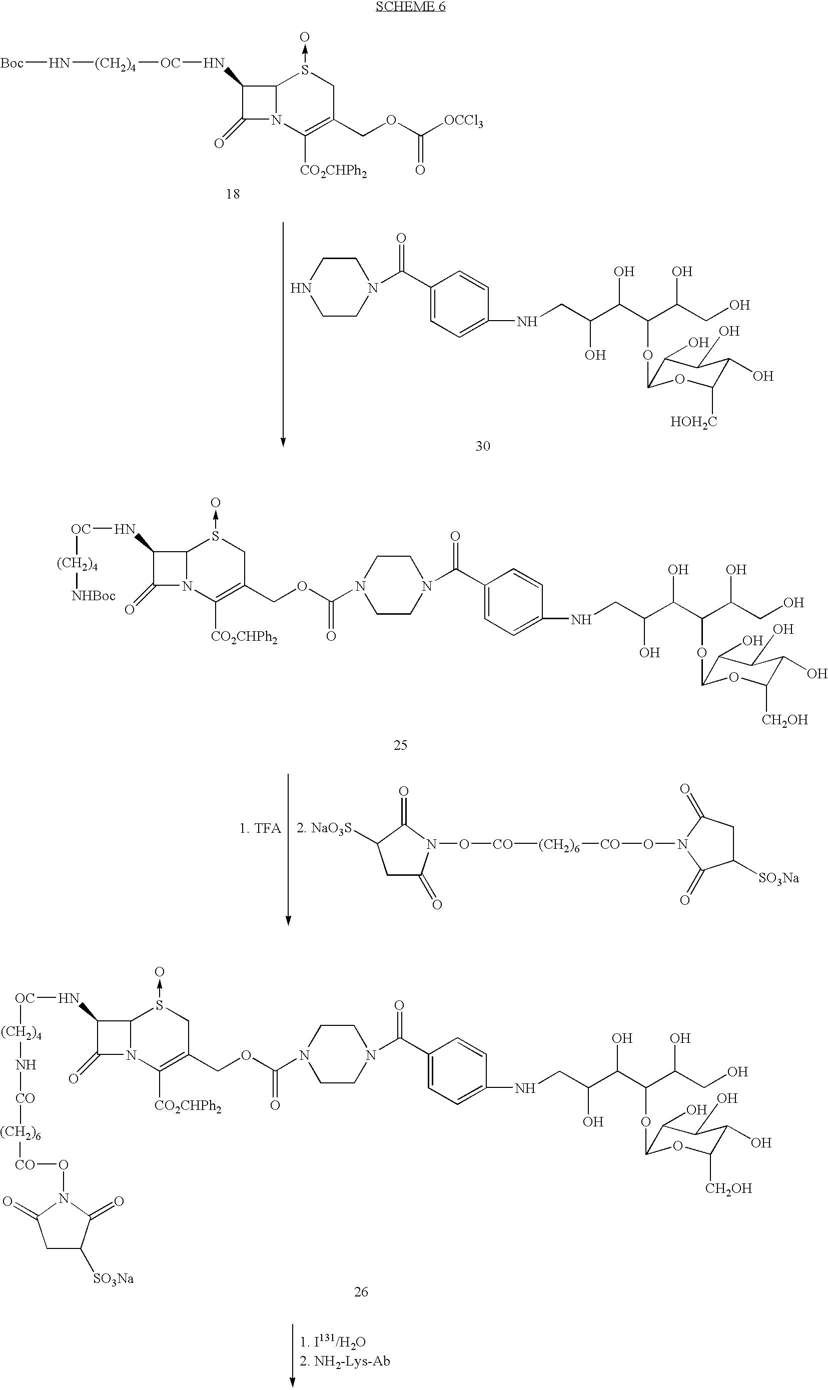

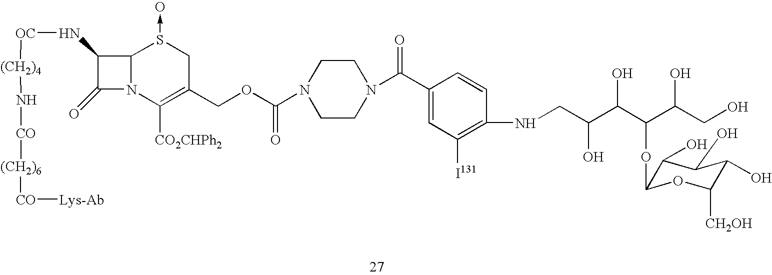

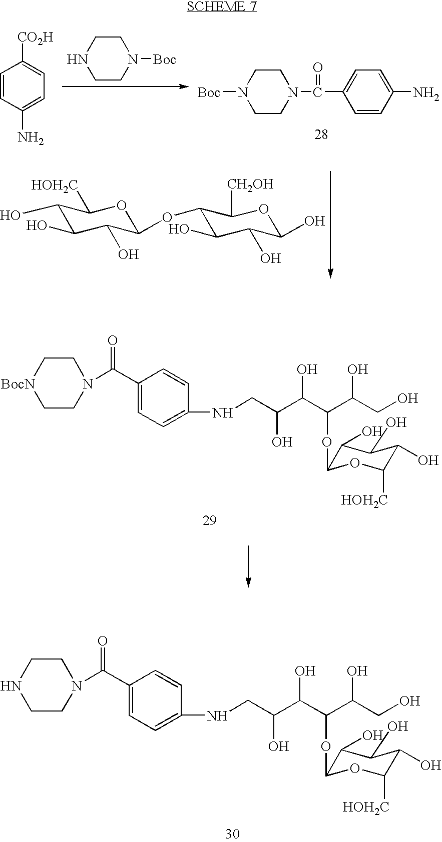

### Example 4

β-Lactamase Sensitivity of Bioconjugates

Compound 14 was radioiodinated using the standard chloramine T method (W. M. Hunter and F. C. Greenwood, Nature, 194:495 (1962); Biochem. J., 89:144 (1963); Proceedings of the Society for Experimental Biology & Medicine, 133(3):989-92, 1970). The reaction mixture was quenched and the resultant product was purified over a C-18 column. The purified product was incubated with B1 anti-CD20 monoclonal antibody (L. M. Nadler, Leukocyte Typing II, Vol. 2, Reinherz et al., eds., New York, Springer-Verlag, 1986; BioGenex Lab., San Ramon, Calif.) (1 hour, room temperature), to yield the corresponding bioconjugate, which was isolated by size-exclusion chromatography, the appropriate isolated fractions were pooled, and the expected size of the conjugate was verified by SDS-PAGE. Immunoreactivity of the bioconjugate was identical to immunoreactivity observed with directly iodinated B1 anti-CD20 antibody.

The sensitivity of the metabolizable linker moiety within the bioconjugate to β-lactamase was determined as follows. The bioconjugate was incubated in the presence (test) and absence of (control) β-lactamase (30 μg), for 30 min at 30 μg/ml at 37° C. The reaction mixture was analyzed by SDS-PAGE, and a marked decrease in radioactivity associated with the antibody was observed. The protein-containing fractions for the test and control reactions were isolated by size exclusion chromatography, and the radioactivity for each fraction was determined. Approximately 85% decrease in radioactivity was observed in the fraction isolated from the test reaction as compared to the fraction isolated from the control reaction, indicating that the metabolizable linker moiety within the bioconjugate was substantially cleaved by the enzyme.

### Example 5

β-lactamase Sensitivity of Bioconjugates

Radiolabeled bioconjugates were synthesized as described in Examples 2 and 3 above, wherein the therapeutically or diagnostically effective agent is I-131; an aryl glycoside such as an iodinated phenyl ring attached to glucose, lactose, cellobiose; or nicotinic acid derivatives, such as iodopyridine carboxylate, 5-iodo-3-pyridinecarboxylate; or metal chelates (DOTA) of radiometals such as Y-90, and the like.

The sensitivity of the metabolizable linker moiety within the bioconjugate β-lactamase was determined as described in Example 4 above. Specifically, bioconjugates of the formula II wherein R¹ is aryl glycoside, 5-iodo-3-pyridinecarboxylate or DOTA conjugated to a radioisotope, and the targeting agent is an internalizing antibody were evaluated. The β-lactamase cleavage eliminates the carrier modified with hexyl amine.

### Example 6

Preparation of Anti-CD20 Antibody Constructs

Various antibody constructs are prepared and tested as follows. Preferably anti-CD20 antibody constructs are used, along with compounds of Structure (II). Examples of constructs suitable for use include, but are not limited to, an anti-CD20 antibody construct where the 1F5 scFv is fused to the C_{H}1 domain, with different sized linkers; a classic scFv with a 15 amino acid linker, that contains a cysteine for chemical cross-linking to the dimer; and a 1F5 “diabody” construct that has a 5 amino acid linker to promote intermolecular diabody formation. These reagents are also used to prepare minibodies.

Construct 1: 1F5scFv-C_{H}1 Antibody Fragments with Varying Linker Lengths

The heavy and light chain variable regions of the murine anti-human CD20 mAb 1F5 are cloned and expressed to optimize the binding properties of 1F5 single chain mAb derivatives (Shan D., et al, J Immunol, 162(11):6589-6595, 1999). Four single chain antibody molecules with a C_{H}1 domain are constructed using linker peptides of variable lengths to join the V_{H} and V_{L} domains of a murine anti-CD20 mAb (1F5). Three constructs are engineered using linker peptides of 15, 10, and 5 amino acid residues consisting of (GGGGS)₃, (GGGGS)₂, and (GGGGS)₁ sequences, respectively, the fourth construct is prepared by joining the V_{H} and V_{L} domains directly. Each construct is fused to a derivative of human IgG1 (hinge+C_{H}2+C_{H}3) by a thrombin-cleavable domain to facilitate purification using staphylococcal protein A, and for the detection of binding activities of these scFvs by anti-human Ig antibodies. The Fc region can be deleted by digestion with thrombin.

The aggregation and CD20 binding properties of these 1F5 scFv-Ig derivatives produced in COS cells is determined. Size-exclusion HPLC analysis and Western blots of proteins subjected to non-reducing SDS-PAGE establish that all of the 1F5 scFv-Ig constructs are monomeric with M.W. of about 55,000. The CD20 binding properties of the 1F5 scFv-Ig constructs are determined by ELISA and flow cytometry techniques. The 1F5 scFv-Ig with the 5 amino acid linker, GS1, demonstrate significantly superior binding to CD20-expressing target cells compared to the rest of the scFv-Ig constructs. The purified GS1 1F5 scFv binds to Ramos target cells, as determined by immunofluorescence and flow cytometry, using a fluoresceinated goat anti-mouse immunoglobulin reagent. Scatchard analysis of radiolabeled GS1 scFv-Ig reveals an estimated binding avidity of 1.35×10⁸ M⁻¹ compared to 7.56×10⁸ M⁻¹ for the native bivalent 1F5 antibody. The GS1 scFv-Ig with a short linker peptide of approximately 5 amino acids is the preferred scFv construct for use in the bioconjugates of the invention.

Construct 2: 1F5scFv with 15 Amino Acid Linker

A true 1F5scFv is constructed by deleting the C_{H}1 gene sequence from the construct described above. This scFv expresses and refolds at excellent levels in a bacterial system. In binding studies, the scFv displays a binding isotherm consistent with specific recognition of the CD20 and minimally reduced affinity relative to the parent antibody.

This construct is used to prepare minibodies and/or a dimeric form of the construct To facilitate cross-linking, the 1F5 scFv is constructed with a cysteine at the C-terminus. The scFv protein is treated with DTT (4 mM) at room temperature (1 h) to yield the dimer. DTT is removed using a PD-10 column pre-equilibrated with Sodium Phosphate (100 mM), EDTA (1 mM) at pH 6.0, and bis-maleimide (0.5 molar equivalent) is added for 30 min. The monomeric and dimeric forms of 1F5 scFv are separated using gel-filtration HPLC, and the size of the protein is determined using SDS-PAGE.

Construct 3: 1F5scFv Diabody

To construct a diabody, the linker is reduced to 5 amino acids to prevent intramolecular association of the V_{H} and V_{L} domains, and to promote intermolecular association to form a diabody. Each V region is separately amplified with specific primers to produce a variable region flanked with a 5 amino acid linker of Ser(Gly)₄. The primary amplification products are purified. A secondary amplification using these products and the two primers which span the entire gene is performed. The sense primer for 1F5 scFv contains an NdeI site upstream of the V_{L} region. The antisense primer for the 1F5 scFv includes a cysteine, 6 histidine residues and two stop codons which are followed by a HindIII restriction site downstream of the V_{H} region. The V_{L} region primer is designed to code for the V_{L} sequence, followed by 5 amino acids of the linker and 18 bases of the 5i of the V_{H} region. The V_{H} primer codes only for the 5 amino acid linker and the V_{H} sequence. The amplified products are extracted from an agarose gel (1.1%), and isolated using a QIAEX II Gel extraction kit (Qiagen GMBH, Hilden, Germany). For the secondary amplification reaction, the sense and antisense primers from the 1F5 scFv construct are used along with 10 μl of each of the isolated V_{L} and V_{H}. The remainder of the diabody construction is identical to that used for the 1F5 scFv.

### Example 7

Cloning of Immunoglobulin V_{H} and V_{L} Domains

Single chain and dimeric anti-CD19 and/or anti-CD22 constructs are made similar to the methods used for preparing anti-CD20 antibody, as described above in Example 6. Methods for cloning and preparing V_{H} and V_{L} domains from hybridoma lines secreting these antibodies are described below.

Immunoglobulin V regions are cloned by RT-PCR mRNA from the respective hybridoma lines HD37 (CD19) and HD39 (CD22), are isolated using Tri-reagent (Sigma Chemical Co., St. Louis, Mo.) and the Qiagen Oligotex RNA isolation kit (Qiagen GMBH, Hilden, Germany). Immunoglobulin mRNA is reverse-transcribed using isotype-specific reverse primers. The cDNA is amplified using a set of oligonucleotides complementary to mouse signal peptide sequences, in combination with the reverse primers. Amplification products are digested with ApaLI and MluI, whose recognition sequences are encoded in the PCR primers and are rarely found in mature immunoglobulin genes (Persic L. et al., Gene, 187:9-18, 1997). The cut fragments are cloned in a derivative of pUC119 that are made with a novel multiple cloning site specifically for PCR cloning of antibody V region genes.

Cloned amplification products are sequenced, and the sequences examined to confirm that they encode valid immunoglobulin genes. The separate V_{H} and V_{L} segments are re-amplified and joined by PCR with a sequence encoding an oligopeptide linker. In addition, a His₅ tag is joined to the C-terminus. The scFv sequences thus formed are subcloned in the E. coli expression vector pAK19 (See Carter P. et al, Bio/Technology, 10:163-167, 1992 and Holmes M. A. et al, J Exp Med, 187:479-485, 1998). Anti-CD19 and/or anti-CD22 scFvs are purified from periplasmic fluid by affinity chromatography on Ni-Sepharose.

### Example 8

Preparation of Minibodies

The following method is used to reconstruct anti-CD19, anti-CD20, and anti-CD22 scFvs as minibodies. The minibodies are constructed according to the method of Hu et al. (Hu S. Z. et al, Cancer Res, 56:3055-3061, 1996), wherein two linkers are used between the scFv equivalent and the CH3 domain of the minibody.

To prepare the minibodies, the His₅ tag sequence of the CD19 and CD22 scFv is deleted. A synthetic sequence encoding the human IgG1 hinge peptide is fused to the C termini of all three scFv's. These constructs are individually subcloned in the expression vector pcDNA3.1neo (Invitrogen Corp., Carlsbad, Calif.). This vector is based on pSV2neo, with the addition of the HCMV-MIE enhancer. The human IgG1 CH3 constant domain exon is added, and the complete construct is transfected by electroporation into NS0 cells. Stable transfectants are selected using G418, and cell clones obtained by limiting dilution. Cell clones secreting high levels of the respective minibodies are identified by sandwich ELISA, using a commercial anti-human IgG Fc for capture and a polyclonal anti-IgG conjugate from the same species for quantitation. The recombinant protein is isolated from the culture medium in which the cells are grown, by affinity chromatography on Protein G-Sepharose. Alternatively, cells are grown in oscillating bubble chambers and isolated by ion exchange and size-exclusion HPLC (see, e.g., Pannell R. and Milstein C., J Immunol Methods, 146:43-48, 1992 and Perkins S. J., Eur J Biochem, 157:169-180, 1986).

Purity of recombinant proteins is ascertained by SDS-PAGE. The molecular weight is determined by electrospray mass spectrometry. Concentration of purified minibodies is quantitated by ultraviolet absorption, based on extinction coefficients calculated from the peptide sequence.

### Example 9

Biodistribution of β-Lactamase-Sensitive Bioconjugates

The in vivo susceptibility of bioconjugates (anti-CD20, anti-CD19 or anti-CD22), prepared as described above, to enzymatic cleavage induced by exogenously administered enzyme, the clearance of the cleaved moiety, and the biodistribution of the radioisotope in tumor and normal organs is determined as follows.

In particular, bioconjugates wherein the targeting agent comprises constructs formed from dimeric or trimeric fragments, such as F(ab′) or scFv fragments, with M.W. greater than 50,000 are used. Anti-CD20 antibody-based constructs are evaluated The anti-CD20 antibody constructs include (i) a construct where the 1F5 scFv has been fused to the C_{H}1 domain with different sized linkers, (ii) a classic scFv with a 15 amino acid linker, that contains a cysteine for chemical cross-linking to the dimer, (iii) a 1F5 diabody construct with a 5 amino acid linker to promote intermolecular diabody formation, and (iv) a minibody, i.e., a dimeric construct containing scFv linked with a C_{H}3 domain. The anti-CD20 constructs are described above, and methods for preparing minibodies are described in Examples 6-8.

The binding characteristics of the antibody constructs are evaluated using Scatchard analysis and FACS assays (see Badger C. C. et al., Nucl Med Biol, 14:605-610, 1987 and Press O.W. et al., Blood, 81:1390-1397, 1994). These constructs are directly radiolabeled and their in vivo biodistribution in tumor-bearing mice is determined. These constructs are then labeled using a β-lactamase-sensitive linker and the effect of enzyme administration on tumor compared to normal tissue radiation is determined as described below.

### Example 10

In vivo Metabolism and Biodistribution of Bioconjugates

Bioconjugates comprising anti-CD20 antibody were evaluated to determine in vivo sensitivity to β-lactamase as follows. The bioconjugate was administered to mice (normal and tumor-bearing mice), followed by infusion of β-lactamase. The extent of cleavage of the bioconjugate and the biodistribution of the radioisotope at various points was determined. In tumor-bearing mice, the biodistribution of radioisotope to tumor as compared to normal tissues post-β-lactamase administration was also determined. Initial experiments demonstrated no differences between the biodistribution of directly iodinated B1 anti-CD20 antibody and the antibody-containing bioconjugate.

The in vivo metabolism of the bioconjugate comprising a β-lactamase-sensitive linker moiety and an anti-CD20 antibody was determined by evaluating blood clearance and urinary excretion in mice (24 mice). The bioconjugate trace-labeled with I-131 (200 μg) was injected (i.v.) into the tail vein of NOD/SCID mice. After 5.5 h, β-lactamase (48 μg) was administered (i.v.) to group I (12 mice), while group II (12 mice) served as control. Blood and urine samples were collected immediately before, and 30 min, 1 h and 14.5 h after β-lactamase administration. Samples were weighed and radioactivity was determined by gamma counting to calculate percent injected dose per gram tissue (% ID/g).

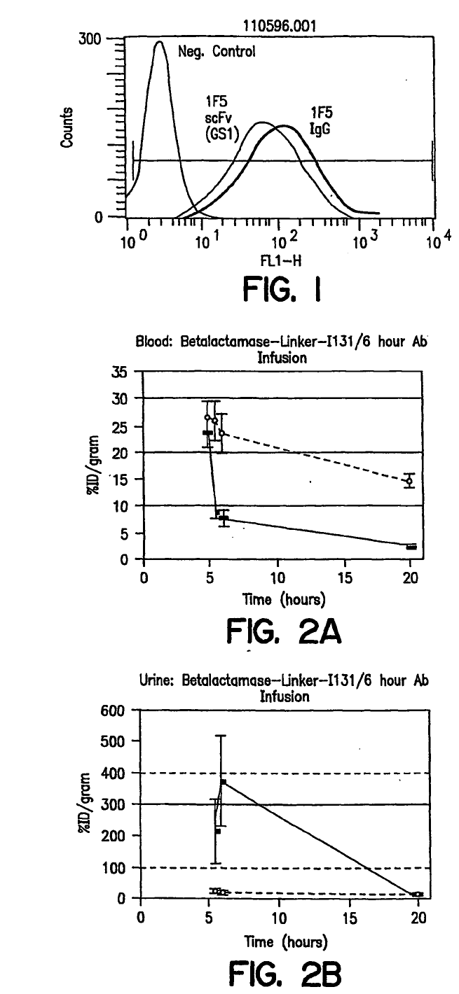

As illustrated in FIGS. 2A and 2B, comparative blood clearance studies between the treated (Group I) and control (Group II) mice, demonstrated a decrease in % ID/g in treated mice—approximately 3-fold decrease at 30 min after enzyme infusion, and a 4-fold decrease at 20 h (FIG. 2A). A 300-fold increase in % ID/g in urine in treated mice was observed 30 min after enzyme infusion (FIG. 2B).

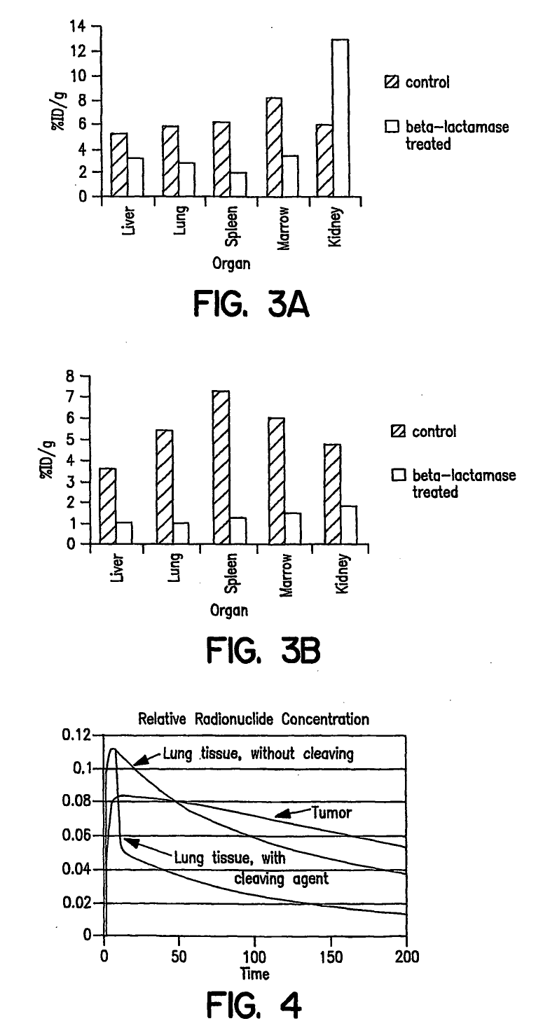

The effect of enzymatic cleavage on radioactive uptake in normal tissues (1 h and 14.5 h) after enzyme infusion was evaluated as follows FIGS. 3A and 3B).

For bone marrow and spleen, a 2-3 fold decrease in % ID/g was observed at 1 h, and 4-6 fold decrease was observed at 14.5 h, in treated versus control mice. For the lung and liver, about a 2-fold decrease in % ID/g was observed at 1 h, and a 3-5 fold decrease was observed at 14.5 h. A 2-fold increase in kidney at 1 h (presumably due to renal clearance of the radioactive moiety) followed by a decrease was observed. These results demonstrate effective in vivo cleavage of the β-lactamase-sensitive linker moiety within the bioconjugate, with rapid removal of the radioisotope by the kidney and a decrease of radioisotope content in the blood, liver, lung and marrow.

### Example 11

In vivo Metabolism and Biodistribution of Bioconjugates

Bioconjugates comprising anti-CD20 antibody were evaluated to determine in vivo sensitivity to pegylated β-lactamase as described above. Pegylated-β-lactamase was tested to determine cleavage of the bioconjugate and retention of the radioisotope at the tumor site, and to decrease the extravascular concentration of the enzyme.

Pegylated-β-lactamase has a M.W. of about 160,000 compared to a M.W. of about 40,000 for the native enzyme. β-lactamase was pegylated with methoxy-PEG-succinimidyl proprionate (M.W. 5000) using standard methods. The enzymatic activity was verified by reaction with nitrocefin (chromogenic cephalosporin substrate), and the molecular weight was assessed by non-reducing SDS PAGE (Zalipsky S. et al., Chem Commun, 653-654, 1999). Higher molecular weight forms of pegylated enzyme were synthesized (Topchieva I. N., Polymer Sci. (USSR), 32:833-851, 1990).

Pegylated β-lactamase retained 78% enzymatic reactivity, migrated at 160 Kd, and cleaved the bioconjugate containing anti-CD20 antibody, in vivo, similar to the native enzyme. Biodistribution studies evaluating pegylated enzyme versus native enzyme, in tumor bearing mice are performed as described below.

A bioconjugate containing an anti-CD20 antibody labeled with 18 μCi I-131 (25 μg) was administered to immunodeficient mice with subcutaneous Ramos B lymphoma cell tumors. Test antibody (400 μg) was also administered to the mice, to decrease nonspecific binding activity. After 20 h, the mice were treated (iv.) with pegylated-β-lactamase (6.4 μg). Mice (3-4 mice group) were sacrificed at 1 h and 4 h post β-lactamase treatment, and organ and tumor samples were collected and weighed. The radioactivity was determined by gamma counting, and percent injected dose per gram tissue (% ID/g) was calculated. The results were as tabulated in Table 1.

TABLE 1
Concentration of radioactivity in normal tissues and tumor
% ID/g (Ratio of tumor:normal tissue % ID/g)

| | 1 Hour | | | | 4 Hour | | | |
|---|---|---|---|---|---|---|---|---|
| | PEG-β- | | | | PEG-β- | | | |
| | lactamase | | No enzyme | | lactamase | | No enzyme | |
| Blood | 5.1 | (1.3)* | 11.9 | (0.6) | 4.5 | (0.9) | 12.1 | (0.6) |
| Marrow | 3.6 | (1.9) | 8.4 | (0.8) | 2.7 | (1.5) | 6.8 | (1.0) |
| Lung | 2.0 | (3.5) | 3.7 | (1.9) | 1.6 | (2.5) | 3.3 | (2.2) |
| Liver | 1.4 | (4.9) | 2.0 | (3.6) | 1.0 | (4.0) | 2.0 | (3.6) |
| Kidney | 4.0 | (1.7) | 2.1 | (3.4) | 2.3 | (1.7) | 2.7 | (2.6) |
| Tumor | 6.9 | | 7.1 | | 4.0 | | 7.1 | |

*Mice were injected with bioconjugate at time 0, Group I received pegylated enzyme at 20 h. Necropsy was performed at 1 h and 4 h (3 mice/group) post enzyme infusion. Tumor:normal tissue % ID/g ratios are shown in parentheses.

Evaluation of organ and tumor distribution demonstrated a greater than 2-fold decrease in blood and marrow radioisotope content at 1 h and 4 h post-pegylated β-lactamase infusion. Significant decrease in radioactive content in lung and liver was also observed, with approximately 2-fold decrease at 4 h post-enzyme infusion. At 1 h, the radioactive content in the kidney increased followed by a decrease at 4 h. In contrast to blood and the normal organs, tumor radioactive content was not significantly decreased at 1 h post infusion of pegylated enzyme. However, by 4 h, the % ID/g had decreased in treated mice. Since bound conjugate is in equilibrium with non-bound conjugate (cleaved and non-cleaved), the decrease in tumor radioactive content was probably due to (i) the exchange of bound labeled bioconjugate with unlabeled bioconjugate and diffusion from the tumor site due to blood clearance, or (ii) cleavage of tumor bound bioconjugate following the delayed entry of the pegylated enzyme into the tumor. A greater than 2-fold increase in tumor:blood ratio of % ID/g at 1 h, and a 50% increase at 4 h was observed for treated versus control mice. The results demonstrate clearance of isotope from normal organs following enzymatic cleavage and improved tumor:normal tissue ratios.

A reduction in tumor radioisotope retention similar to that observed in normal tissues 1 h after enzyme infusion was observed. Comparative studies between pegylated and native enzyme indicated no significant differences in blood and normal organ radioisotope concentration at 1 h and 4 h after enzyme infusion.

### Example 12

In Vivo Metabolism and Biodistribution of Bioconjugates

Internalizing antibodies are preferred because after internalization they are not susceptible to enzymatic cleavage and, are not be available for exchange with extracellularly cleaved bioconjugate (Stein R., et al., J. Nucl. Med., 38:391-395, 1997; Sharkey R. M., et al., Cancer Immunol. Immunother, 44:179-188, 1997.; van der Jagt R. H. C., et al., Cancer Res, 52:89-94, 1992; Press O. W., et al., Blood, 81:1390-1397, 1994; Press O. W., et al., Cancer Res, 56:2123-2129, 1996; Naruki Y., et al., Nucl Med Biol, 17:201-207, 1990). Conventionally iodinated anti-CD19 antibody is internalized by lymphoma cells in vitro, iodine is subsequently secreted by the cell (Press O. W., et al., Blood, 81:1390-1397, 1994). Administration of bioconjugates containing the internalizing anti-CD33 antibody labeled with a radiometal, results in retention of the radioisotope in vivo.

Bioconjugates containing antibodies internalized by target lymphoma cells are tested for internalization and retention of radioisotope in vivo. Bioconjugates comprising a HD37 anti-CD19 antibody, a B4 anti-CD19 antibody, a HD39 anti-CD22 antibody or a MB-1 anti-CD37 antibody, are evaluated to determine in vitro intracellular uptake and retention of the radioisotope by tumor cells.

Specifically, internalization studies are conducted using bioconjugates containing anti-CD19 antibody iodinated using chloramine T. Biodistribution studies in tumor-bearing mice are performed as described above. The extent of catabolism is determined by assessing serum samples and protein precipitated with trichloroacetic acid (TCA) for separation of free and protein bound radioiodine, and measure the radioactive content of the thyroid and stomach. Significant intracellular degradation occurs, with decreased tumor residence time of the I-131, increased free radioiodine clearance from the blood and a higher percentage of free iodide species contributing to blood radioiodine content. Secretion of free radioiodine present in the stomach, colon, and thyroid is increased.

The optimal time points for enzymatic cleavage of bioconjugates of formula (II), wherein R¹ is an aryl glycoside, 5-iodo-3-pyridinecarboxylate, or DOTA conjugated to a radioisotope, is determined as described below in Examples 13 and 16.

### Example 13

In Vivo Metabolism and Biodistribution of Bioconjugates Comprising an Interalizing Antibody in Normal Mice

The biodistribution studies of a bioconjugate is performed using 6-10 week-old BALB/c or C57BL/6 mice (3 sets of 4-5 animals/group). The bioconjugate is administered to each mouse. The mice are divided into two groups: (1) control group and (2) test group. β-lactamase is administered to the test group at 6 h or at 24 h after administration of the bioconjugate. Biodistribution studies are performed before, and 30 minutes, 1 h, 2h, 4 h, 8 h, 24 h, and 48 h after enzyme infusion. Normal tissues and blood are weighed and the radioactive content is determined by comparison with a standard aliquot of the injectate. To determine the extent of cleavage of circulating antibody, the antibody is isolated from the serum using protein G columns and the radioactivity is measured (cpm/mg of protein at OD₂₈₀). Urine is collected and assessed for radioactivity to determine the extent of metabolism of the bioconjugate by the kidneys.

Increased cleavage of bioconjugates, with a greater reduction of radioisotope concentration in blood and normal organs, occurs following administration of increased, or repetitive doses, of native enzyme. This effect is enhanced by administration of pegylated enzyme, as described below. Bioconjugates that are effectively cleaved in vivo with the isotope-containing moiety cleared via the kidney are further studied in tumor-bearing mice.

### Example 14

In Vivo Metabolism and Biodistribution of Bioconjugates Comprising an Internalizing Antibody in Tumor-Bearing Mice

The difference in tumor residence time and normal organ clearance of radioisotope delivered by a bioconjugate in mice, is determined as described above. First, optimal time points for infusion of β-lactamase are estimated by performing biodistribution studies without infusing enzyme in tumor bearing mice. The enzyme is then administered at one or more time points estimated to be optimal for enzyme infusion, as determined in modeling studies performed by Darrell Fischer using the antibody compartment model (Battelle Pacific Northwest National Laboratory). Male or female NOD/SCID mice (6-10 weeks old, 23-27 g) are housed at less than 6 mice per unit in a pathogen-free environment A single cell suspension of the Ramos B cell lymphoma cell line (0.2 mL 10⁷ cells) is administered subcutaneously (s.c) to the mice. The tumor is allowed to grow to 0.3 cm² in size at which time mice are used for biodistribution studies. The relative biodistribution of cleaved or uncleaved bioconjugate is compared to determine the difference in residence time in tumor versus normal organs, using methods described above. A high antibody dose in combination with ECIA is advantageous (3200 μg is required for saturating target cells with a tumor mass) (Sgouros G., J Nucl Med, 33:2167-2179, 1992). The dose of bioconjugate optimal for tumor retention is also determined by administering varying amounts of the bioconjugate, i.e., at 25, 100, 400, 800, and 3200 μg. (Badger C. C., and Bernstein I. D., J Exp Med, 157: 828-842, 1983).

The radiation doses delivered to the tumor and normal tissues is then estimated. Mouse organs are relatively small compared to the range of beta particles. Thus, absorbed fractions and associated “S” values (the absorbed dose/unit cumulated activity) for humans cannot be directly applied to animal dosimetry. To avoid the possibility of cross-organ irradiation component in mice, a separate dosimetry is used to calculate cross-organ beta doses in mice (Hui T. E., et al., Cancer, 73:951-957, 1994). Absorbed fractions of beta energy are calculated using Berger point kernels and electron transport code EGS4, using a computer program developed from the model for calculating radiation doses in the mouse.

As illustrated in FIG. 4, the uptake of radiolabeled antibody is almost instantaneous in normal tissue, whereas the uptake by tumor tissue is somewhat delayed. However, the slope of the time-activity curve for normal tissue is steeper than that of the curve for tumor tissue, which over time indicates a higher radiation absorbed dose for tumors compared to the limiting normal tissue, indicating a favorable therapeutic ratio. The curve representing the time-activity curve for normal tissue after administration of cleaving agent (approximately six hours after antibody injection) results from separation of the radiolabel from the antibody and rapid clearance of the radiolabel from the body via urinary excretion The reduced area-under-curve for normal tissue improves the therapeutic tumor:normal-tissue ratio by 50%. These results suggest the effectiveness of β-lactamase-sensitive linkers and suitably labeled internalizing antibody.

### Example 15

Effect of Pegylated β-Lactamase on the Metabolism and Biodistribution of Bioconjugates Comprising an Internalizing Antibody

A broad range of enzyme doses (0.5, 5, 50 and 500 μg) was tested and the extent of intravascular cleavage, and reduction in % ID/g in blood and normal organs was determined. The effect of multiple dose administration of enzyme on blood enzyme concentration level, and reduction in organ % ID/g was determined. The in vivo half life of radioiodinated enzyme was ascertained to determine an appropriate schedule for multiple dose administration

The effect of pegylated enzyme on the reduction of undesirable cleavage at the tumor site was determined. Generally, a reduction of tumor penetration does not prevent loss of radioisotope from conjugates bound to the tumor cell surface. However, delaying entry of enzyme into the tumor, may permit a delay in local cleavage and, for internalizing antibody, allow greater time for internalization prior to cleavage. Additionally, pegylated enzyme formulations potentially have reduced immunogenicity (Zalipsky, S., Bioconjugate Chem., 6:150-165, 1995 and Dreborg S. and Akerblom E. B., Crit Rev Ther Drug Carrier Syst, 6:315-365, 1990).

The effect of pegylation of β-lactamase on the extent of penetration into the tumor site was determined by radioiodinating the enzyme and assessing the in vivo biodistribution of the radioisotope. The effect of native β-lactamase versus pegylated-enzyme on tumor residence time of a bioconjugate was determined. β-lactamase was pegylated by standard methods with methoxy-PEG-succinimidyl proprionate (M.W. 5000), enzymatic activity was verified by reaction with nitrocefin (chromogenic cephalosporin substrate), and the molecular weight was assessed by non-reducing SDS PAGE (Zalipsky S. et al., Chem Commun, 653-654, 1999).

Pegylated β-lactamase retained 78% enzymatic reactivity, migrated at 160 Kd, and cleaved the bioconjugate containing anti-CD20 antibody, in vivo, similar to the native enzyme. Biodistribution studies evaluating pegylated enzyme versus native enzyme, in tumor bearing mice were performed as described above. Higher molecular weight forms of pegylated enzyme were synthesized (Tropchieva I. N., Polymer Sci. (USSR), 32:833-851, 1990), and their effect on delay or prevention of entry into the extravascular space, and on isotope loss from the tumor due to cleavage was determined.

### Example 16

Biodistribution of Bioconjugates in Non-Human Primates

The in vivo biodistribution of bioconjugates in nonhuman primates is determined as follows. The animals are administered (i) the bioconjugate and the β-lactamase (Group I, 3 animals), and (ii) bioconjugate (Group II, 3 animals), and the time-activity curves for lung, liver, and lymph nodes is evaluated.

Animals are administered bioconjugate trace-labeled with 2 mCi of 1-131 (1.7 mg/kg) and undergo serial quantitative gamma camera imaging at the end of infusion, immediately before and 30 min following β-lactamase infusion, and then daily for 2 days. To assess marrow and lymph node uptake, biopsies are performed immediately before, and 6 h and 24 h post infusion of β-lactamase. Microdistribution of antibody in lymph node tissue is determined by autoradiography (see Clark E. A. and Draves K. E., Eur J Immunol, 17:1799-1805, 1987).

The initial uptake and clearance of radionuclide in lung, liver, and marrow in the animals in Groups I and II is determined

Thus, a bioconjugate comprising a targeting agent conjugated to a diagnostically or therapeutically effective agent by a metabolizable linker moiety, which is cleaved by an exogenous enzyme, is disclosed. Although preferred embodiments of the invention have been described in some detail, it is understood that obvious variations can be made without departing from the spirit and scope of the invention as defined by the appended claims.

We claim:

1. A bioconjugate composition comprising a targeting agent conjugated to a diagnostically or therapeutically effective agent by a metabolizable linker moiety, which is cleaved by an exogenous enzyme.

2. The bioconjugate composition of claim 1 wherein the metabolizable linker moiety is a β-lactamase-sensitive linker moiety.

3. The bioconjugate composition of claim 2 wherein the targeting agent is an antibody.

4. The bioconjugate composition of claim 3 wherein the antibody is an anti-CD19 antibody, an anti-CD20 antibody, an anti-CD22 antibody, an anti-CD33 antibody, an anti-CD37 antibody or an anti-CD45 antibody.

5. The bioconjugate composition of claim 2 wherein the diagnostically or therapeutically effective agent is a radioisotope.

6. The bioconjugate composition of claim 5 wherein the diagnostically or therapeutically effective agent is I-131, iodinated(I-131) aryl glycoside, 5-iodo(I-131)-3-pyridinecarboxylate, Y-90 within metal chelates.

7. The bioconjugate composition of claim 2 comprising the formula (I): 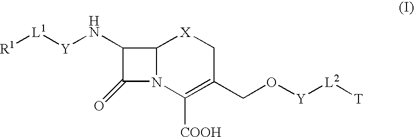
wherein m is an integer ranging from 1 to 12 inclusive; and n is an integer ranging from 1 to 12 inclusive;
L¹ is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —(CHR²)ₙ—CH₂—O—; —(CHR²)ₙ—; —NH—(CHR²)ₙ—NH—; —NH—(CHR²)ₙ—NH—CO—(CHR²)ₘ—CO-Z-; —(CHR²)ₙ—NH—CS—NH—(CHR²)ₘ—CS—NH-Z; —NH—(CHR²)ₙ—NH—CS—(CHR²)ₘ—CO-Z-; —(CHR²)ₙ—NH—CO—NH—(CHR²)ₘ—CO—NH-Z; or a biodegradable polyamino acid macromolecular carrier, wherein L¹-Y—NH taken together optionally form a heterocyclic or a heteroaryl ring;
L² is —(CHR²)ₙ—NH—(CHR²)ₘ—CO-Z; —(CHR²)ₙ—NH—CO—(CHR²)ₘ—CO-Z; —(CHR²)ₙ—NH—; —(CHR²)ₙ—CH₂—S—; —NH—(CHR²)ₙ—NH—; —NH—(CHR²)ₙ—(CHR³)—NH—; —NH—(CHR²)ₙ—NH—CO—(CHR²)ₘ—CO-Z-; —NH—(CHR²)ₙ—NH—CO—(CHR²)ₘ—CO—; —NH—(CHR²)ₙ—NH—CS—(CHR²)ₘ—CO-Z-; —NH—(CHR²)ₙ—NH—CS—(CHR²)ₘ—CO—; —(CHR²)ₙ—CH₂—O—; —(CHR²)ₙ—; or a biodegradable polyamino acid macromolecular carrier; wherein L² optionally forms cyclic structure comprising an aryl ring, heteroaryl ring, cycloalkyl ring, cycloalkenyl ring, wherein said ring is optionally substituted;
T is a targeting agent;
X is O, NH, S or SO;
Y is CO or CS;
Z is an amino acid, N-hydroxysuccinimydl (NHS) or sulfonated N-hydroxysuccinimydl;
R¹ is a diagnostically or therapeutically effective agent;
R² is H, OH, lower alkyl, alkoxy, acyloxy, alkylamino, alkylthio or hydroxyalkyl;
R³ is —COOH or —CH₂OSO₃H; or
a pharmaceutically acceptable salt thereof.

8. The bioconjugate composition of claim 2 comprising the formula (II): 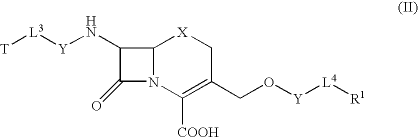
wherein m is an integer ranging from 1 to 12 inclusive; and n is an integer ranging from 1 to 12 inclusive;
L³ is —(CHR²)ₙ—NH—(CHR²)ₘ—CO-Z; —(CHR²)ₙ—NH—CO—(CHR²)ₘ—CO-Z; —(CHR²)ₙ—CO—NH-Z; —(CHR₂)ₙ—NH—; —(CHR²)ₙ—NH—CO—NH—(CHR²)ₘ—CO—NH-Z-; —(CHR²)ₙ—CH₂—S—; —(CHR²)ₙ—CH₂—O—; —NH—(CHR²)ₙ—NH—CS—(CHR²)ₘ—CO-Z; —NH—(CHR²)ₙ—NH—; —NH—(CHR²)ₙ—NH—CO—(CHR²)ₘ—CO-Z; —(CHR₂)ₙ—; —(CHR²)ₙ—NH—CS—NH—(CHR²)ₘ—CS—NH-Z; or a biodegradable polyamino acid macromolecular carrier, wherein L³-Y—NH taken together optionally form a heterocyclic or a heteroaryl ring;
L⁴ is —(CHR²)ₙ—NH—(CHR²)ₘ—CO-Z; CHR²)ₙ—NH—CO—(CHR²)ₘ—CO-Z; —(CHR²)ₙ—NH—; —(CHR²)ₙ—CH₂—S—; —NH—(CHR²)ₙ—NH—CO—(CHR²)ₘ—CO-Z-; —(CHR²)ₙ—CH₂—O—; —(CHR²)ₙ—; —NH—(CHR²)ₙ—NH—; —NH—(CHR)ⁿ—(R³)—NH—; —NH—(CHR²)ₙ—NH—CO—(CHR²)ₘ—CO—; —NH—(CHR³)ₙ—NH—CS—(CHR²)ₘ—CO-Z-; —NH—(CHR²)ₙ—NH—CS—(CHR²)ₘ—CO—; or a biodegradable polyamino acid macromolecular carrier, wherein L⁴ optionally forms cyclic structure comprising an aryl ring, heteroaryl ring, cycloalkyl ring, cycloalkenyl ring, wherein said ring is optionally substituted;
T is a targeting agent;
X is O, NH, S or SO;
Y is CO or CS;
Z is an amino acid, N-hydroxysuccinimydl (NHS) or sulfonated N-hydroxysuccinimydl;
R¹ is a diagnostically or therapeutically effective agent;
R² is H, OH, lower alkyl alkoxy, acyloxy, alkylamino, alkylthio or hydroxyalkyl;
R³ is —COOH or —CH₂OSO₃H; or
a pharmaceutically acceptable salt thereof.

9. The bioconjugate composition of claim 7 or claim 8 wherein T is an antibody.

10. The bioconjugate composition of claim 9 wherein T is an anti-CD19 antibody, an anti-CD20 antibody, an anti-CD22 antibody, an anti-CD33 antibody, an anti-CD37 antibody or an anti-CD45 antibody.

11. The bioconjugate composition of claim 7 or claim 8 wherein R¹ is a radioisotope.

12. The bioconjugate composition of claim 11 wherein the diagnostically or therapeutically effective agent is I-131, iodinated(I-131) aryl glycoside, 5-iodo(I-131)-3-pyridinecarboxylate, Y-90 within metal chelates.

13. The bioconjugate composition of claim 7 comprising the formula (I-A) 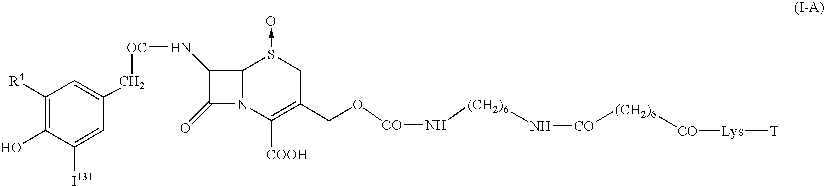
wherein T is an antibody, biotin, streptavidin or avidin; and R⁴ is H or I¹³¹.

14. The bioconjugate composition of claim 8 comprising the formula (II-A) 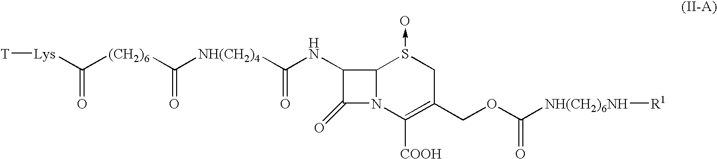
wherein T is an antibody, biotin, streptavidin or avidin; and
R¹ is an iodinated(I-131) aryl glycoside, 5-iodo(I-131)-3-pyridinecarboxyl or Y-90 1,4,7,10-tetraazacyclododecane-N,N′,N″,N′″-ttetraacetic acid (DOTA) complex.

15. The bioconjugate composition of claim 8 comprising the formula (II-C) 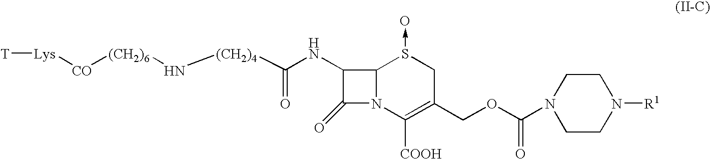
wherein T is an antibody, biotin, streptavidin or avidin; and
R¹ is an iodinated(I-131) aryl glycoside, 5-iodo(I-131)-3-pyridinecarboxyl or Y-90 1,4,7,10-tetraazacyclododecane-N,N′,N″,N′″-tetraacetic acid (DOTA) complex.

16. A method for treating a disease comprising administering to a mammal in need of such treatment a pharmaceutically effective amount of a bioconjugate according to claim 1, and a pharmaceutically effective amount of an enzyme capable of cleaving said metabolizable linkage.

17. The method of claim 16 wherein the enzyme is administered subsequent to administering the bioconjugate.

18. The method of claim 16 wherein the metabolizable linker moiety is a β-lactamase-sensitive linker moiety.

19. The method of claim 18 wherein the enzyme is β-lactamase.

20. The method of any one of claims 16-19 wherein the targeting agent is an antibody.

21. The method of claim 20 wherein the antibody is an anti-CD19 antibody, an anti-CD20 antibody, an anti-CD22 antibody, an anti-CD33 antibody, an anti-CD37 antibody or an anti-CD45 antibody.

22. The method of any one of claims 16-19 wherein the diagnostically or therapeutically effective agent is a radioisotope.

23. The method of claim 22 wherein the diagnostically or therapeutically effective agent is I-131, iodinated(I-131) aryl glycoside, 5-iodo(I-131)-3-pyridinecarboxylate, Y-90 within metal chelates.

24. A method for the delivery of a diagnostic or a therapeutically effective agent to cells comprising:
administering a pharmaceutically effective amount of a bioconjugate according to claim 1, wherein said targeting agent is reactive with a binding site on the surface of said cells; and
administering a pharmaceutically effective amount of an enzyme capable of cleaving said metabolizable linkage.

25. The method of claim 24 wherein the enzyme is administered subsequent to administering the bioconjugate.

26. The method of claim 24 wherein the metabolizable linker moiety is a β-lactamase-sensitive linker moiety.

27. The method of claim 26 wherein the enzyme is β-lactamase.

28. The method of any one of claims 24-27 wherein the targeting agent is an antibody.

29. The method of claim 28 wherein the antibody is an anti-CD19 antibody, an anti-CD20 antibody, an anti-CD22 antibody, an anti-CD33 antibody, an anti-CD37 antibody or an anti-CD45 antibody.

30. The method of any one of claims 24-27 wherein the diagnostically or therapeutically effective agent is a radioisotope.

31. The method of claim 30 wherein the diagnostically or therapeutically effective agent is I-131, iodinated(I-131) aryl glycoside, 5-iodo(I-131)-3-pyridinecarboxylate, Y-90 within metal chelates.

32. A method of detecting the presence of a disease in a mammal suspected of having a said disease, comprising administering to the mammal a diagnostically effective amount of a bioconjugate according to claim 1, and an effective amount of an enzyme capable of cleaving said metabolizable linkage.

33. The method of claim 32 wherein the enzyme is administered subsequent to administering the bioconjugate.

34. The method of claim 32 wherein the metabolizable linker moiety is a β-lactamase-sensitive linker moiety.

35. The method of claim 34 wherein the enzyme is β-lactamase.

36. The method of any one of claims 32-35 wherein the targeting agent is an antibody.

37. The method of claim 36 wherein the antibody is an anti-CD19 antibody, an anti-CD20 antibody, an anti-CD22 antibody, an anti-CD33 antibody, an anti-CD37 antibody or an anti-CD45 antibody.

38. The method of any one of claims 32-35 wherein the diagnostically or therapeutically effective agent is a radioisotope.

39. The method of claim 38 wherein diagnostically or therapeutically effective agent is I-131, iodinated(I-131) aryl glycoside, 5-iodo(I-131)-3-pyridinecarboxylate, Y-90 within metal chelates.

40. The bioconjugate composition of claim 7 wherein the amino acid is selected from the group consisting of lysine, serine, threonine, tyrosine and cysteine.

41. The bioconjugate composition of claim 8 wherein the amino acid is selected from the group consisting of lysine, serine, threonine, tyrosine and cysteine.
